(19) United States
(12) Patent Application Publication
(10) Pub. No.: US 2011/0294833 A1
(43) Pub. Date: Dec. 1, 2011

(54) Methods for Healthy Aging

(75) Inventors: Eric Topol, La Jolla, CA (US); Sarah Murray, Encinitas, CA (US); Bradley Patay, San Diego, CA (US)

(73) Assignee: SCRIPPS HEALTH, La Jolla, CA (US)

(21) Appl. No.: 12/789,021

(22) Filed: May 27, 2010

Related U.S. Application Data
(60) Provisional application No. 61/348,651, filed on May 26, 2010.

(51) Int. Cl. A61K 31/519; A61P 25/28; A61P 9/00; A61P 19/10; A61P 27/00; A61P 3/10; A61P 3/04

(52) U.S. Cl. 514/262.1

(57) ABSTRACT

Genome-wide association studies have been used to elucidate genes and/or pathways related to diseases; however, this methodology has yet to be used to understand the phenotype of healthy aging and/or healthspan in humans.

### CROSS-REFERENCE

This application claims the benefit of U.S. Provisional Application No. 61/348,651, filed May 26, 2010, which is incorporated herein by reference in its entirety.

### STATEMENT AS TO FEDERALLY SPONSORED RESEARCH

This invention was made with the support of the United States government under Contract number NCRR/CTSA UL1 RR025774, RO1 AG005407, U01 AG018197 and U19 AG023122 by National Institutes of Health.

### BACKGROUND OF THE INVENTION

There is interest in methods of promoting healthy aging and/or extending disease-free lifespan in humans.

### SUMMARY OF THE INVENTION

Described herein are the associations of certain genomic regions in humans with a phenotype of healthy aging and/or a delay in onset of chronic age-related diseases or conditions such as cardiovascular disease, metabolic syndrome, bone-loss disorder and/or neurodegenerative disorders. Also described herein are the uses of such associations for promoting healthy aging in humans. Also described herein are methods and compositions for promoting healthy aging in humans, including, e.g. as in the wellderly population. Also provided herein are methods and compositions for preventing or delaying the onset and/or progression of diseases associated with the illderly population.

Provided herein are methods of promoting healthy aging in a human in need thereof or preventing a disease associated with the elderly in a human in need thereof comprising administering to the human an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof.

In some embodiments, the disease associated with the elderly is selected from a cardiovascular disease, a metabolic syndrome, a bone-loss disorder or a neurodegenerative disease.

In some embodiments, the disease associated with the elderly is selected from the group consisting of pre-diabetes, diabetes, obesity, osteoporosis, coronary artery disease, cerebrovascular disease, heart attack, stroke, peripheral arterial disease, aortic valve disease, stroke, mild cognitive impairment, pre-dementia, dementia, macular degeneration, and cataracts.

In some embodiments, the sequence responsible for the expression of a gene contains at least one illderly- or wellderly-associated single nucleotide polymorphism (SNP).

In some embodiments, the illderly- or wellderly-associated SNP is found in the region spanning from genes SLC4A9 to SLC35A4, the region encompassing the gene APPB3, the region encompassing the gene ANKHD1-EIF4EBP3, the region encompassing the gene EIF4BP3, the region encompassing the gene ANKHD1, the region encompassing the gene SLC4A9, the region encompassing the gene SLC35A4, or the region encompassing the gene SRA1.

In some embodiments, the illderly- or wellderly-associated SNP is selected from the group consisting of rs1862176, rs6879217, rs34507975, rs6860077, rs11168051, rs17286676, rs13171154, rs35309382, rs6872579, rs7356554, rs975243, rs1432959, rs12717986, rs10042299, rs12717988, rs7715360, rs7701365, rs3822328, rs10476907, rs1019933, rs10041762, rs9687753, rs12109242, rs6876865, rs6871703, rs7712617, rs6579768, rs3733681, rs9324644, rs250432, rs250424, rs250425, rs801460, rs801459, rs10463297, rs250426, rs2249213, rs250431, rs250430, rs250429, rs2253378, rs4912715, rs801458, rs4279384, rs1864255, rs2569163, rs1835148, rs7444290, rs778583, rs778584, rs7721577, and rs778587.

In some embodiments, the illderly- or wellderly-associated SNP is selected from the group consisting of rs250431, rs2071004, rs1863291, rs7609512, rs7016232, rs10758570, rs10866584, rs17620029, rs4765461, rs6903798, rs1386267, rs5971801, rs3749898, rs11590076, rs5927244, rs2889450, rs7815797, rs1929363, rs8095607, rs755993, rs1051309, rs3172957, and rs3752704.

In some embodiments, the at least one gene is selected from the group consisting of SLC4A9, ANKHD1-EIF4EBP3, APPB3, ANKHD1, EIF4EBP3, SRA1 and SLC35A4.

In some embodiments, the at least one gene is EIF4EBP3.

In some embodiments, the at least one expressed product is a protein. In some embodiments, the protein is a 4E-BP. In some embodiments, the protein is selected from the group consisting of EIF4BP3 (NP_{—}003723.1), ANKHD1-EIF4EBP3 (NP_{—}065741.3), ANKHD1 (NP_{—}060217.1, NP_{—}060448.1, NP_{—}078944.2), APPB3 (NP_{—}006042.3, NP_{—}573418.2, NP_{—}573419.2, NP_{—}573420.2), SLC4A9 (NP_{—}113655.2), SRA1 (NP_{—}001030312.2), and SLC35A4 (NP_{—}542401.1).

In some embodiments, the protein is a functional protein. In some embodiments, the protein is a non-functional protein.

In some embodiments, the at least one expressed product is an RNA.

In some embodiments, the agent increases or promotes binding of a eukaryotic translation initiation factor 4E-binding protein (4E-BP) to eukaryotic translation intitiation factor 4E (eIF4E). In some embodiments, the agent that increases or promotes binding of a 4E-BP to eIF4E binds to an allosteric site of the 4E-BP.

In some embodiments, the agent increases binding of eukaryotic initiation factor (4E-BP) to eukaryotic initiation factor 4E (eIF4E) to reduce cap-dependent translation or increase nuclear sequestration of eIF4E. In some embodiments, the agent disrupts binding of eIF4E to eIF4G to decrease cap-dependent translation or increases nuclear sequestration of eIF4E.

In some embodiments, the agent mimics a 4E-BP hypophosphorylated state and competes with eIF4G in binding to eIF4E to form eIF4F.

In some embodiments, the 4E-BP is 4E-BP3.

In some embodiments, the agent is a 4E-BP3 blocker.

In some embodiments, the agent
(a) reduces or inhibits phosphorylation of 4E-BP3; and/or
(b) increases or promotes binding of 4E-BP3 to eIF4E.

In some embodiments, the agent that reduces or inhibits phosphorylation of a 4E-BP is an inhibitor or partial inhibitor of a kinase selected from the group consisting of Akt/PI-3 kinase, mTOR kinase, a MAP kinase, a S6 kinase, and Cdc2 kinase. In some embodiments, the agent that reduces or inhibits phosphorylation of 4E-BP3 is an inhibitor or partial inhibitor of a kinase selected from the group consisting of Akt/PI-3 kinase, mTOR kinase, a MAP kinase, a S6 kinase, and Cdc2 kinase.

In some embodiments, the agent is administered in combination with a modulator of IGF, FOXO3, AKT, TOR, or Sirtuin.

In some embodiments, the agent is administered in combination with a sirtuin activator. In some embodiments, the agent is administered in combination with a S6K inhibitor. In some embodiments, the agent is administered in combination with a modulator of the IGF pathway. In some embodiments, the agent is administered in combination with an mTOR kinase inhibitor. In some embodiments, the agent is administered in combination with a PI-3 kinase inhibitor.

In some embodiments, the agent is administered in combination with an agent that modulates the activity or expression of at least one gene in the 4EIFBP1 or 4EIFBP2 genomic region, or (b) modulates the level or activity of at least one expressed product thereof.

In some embodiments, the human in need thereof has a SNP selected from rs1862176, rs6879217, rs34507975, rs6860077, rs11168051, rs17286676, rs13171154, rs35309382, rs6872579, r57356554, rs975243, rs1432959, rs12717986, rs10042299, rs12717988, rs7715360, rs7701365, rs3822328, rs10476907, rs1019933, rs10041762, rs9687753, rs12109242, rs6876865, rs6871703, rs7712617, rs6579768, rs3733681, rs9324644, rs250432, rs250424, rs250425, rs801460, rs801459, rs10463297, rs250426, rs2249213, rs250431, rs250430, rs250429, rs2253378, rs4912715, rs801458, rs4279384, rs1864255, rs2569163, rs1835148, rs7444290, rs778583, rs778584, rs7721577, and rs778587.

In some embodiments, the human in need thereof has a SNP selected from the group consisting of rs250431, rs2071004, rs1863291, rs7609512, rs7016232, rs10758570, rs10866584, rs17620029, rs4765461, rs6903798, rs1386267, rs5971801, rs3749898, rs11590076, rs5927244, rs2889450, rs7815797, rs1929363, rs8095607, rs755993, rs1051309, rs3172957, and rs3752704.

In some embodiments, the human has an aberrant and/or over-expressed product of the genomic region EIF4EBP3.

In some embodiments, the disease associated with the elderly is diabetes. In some embodiments, the agent prevents or delays the transition from a pre-diabetes condition to diabetes. In some embodiments, the disease associated with the elderly is osteoporosis. In some embodiments, the agent prevents or delays the transition from osteopenia to osteoporosis. In some embodiments, the disease associated with the elderly is dementia. In some embodiments, the agent prevents or delays coronary artery disease, cerebrovascular disease, heart attack, stroke, peripheral arterial disease, or aortic valve disease. In some embodiments, the agent prevents or delays macular degeneration or cataracts.

In some embodiments, the agent is a compound having structure:

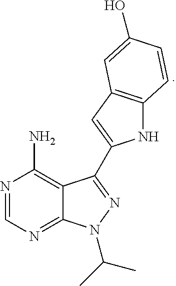

Also provided herein is a method for predicting a risk for unhealthy aging in a human comprising
(a) detecting, in a sample obtained from said human, at least one illderly-associated single nucleotide polymorphism (SNP), in the genomic region spanning from position 139710628 to 139986614 base pairs on human chromosome 5; and
(b) correlating the detection of the at least one illderly-associated SNP in said human to risk of unhealthy aging;

wherein said detecting is carried out on a Genome Analyzer.

In some embodiments, the illderly-associated SNP is in the genomic region EIF4EBP3. In some embodiments, the illderly-associated SNP is one or more of SNPs described in Table 2, Table 3, Table 4, or Table 5. In some embodiments, the illderly-associated SNP is one or more of rs1862176, rs6879217, rs34507975, rs6860077, rs11168051, rs17286676, rs13171154, rs35309382, rs6872579, rs7356554, rs975243, rs1432959, rs12717986, rs10042299, rs12717988, rs7715360, rs7701365, rs3822328, rs10476907, rs1019933, rs10041762, rs9687753, rs12109242, rs6876865, rs6871703, rs7712617, rs6579768, rs3733681, rs9324644, rs250432, rs250424, rs250425, rs801460, rs801459, rs10463297, rs250426, rs2249213, rs250431, rs250430, rs250429, rs2253378, rs4912715, rs801458, rs4279384, rs1864255, rs2569163, rs1835148, rs7444290, rs778583, rs778584, rs7721577, and rs778587.

In some embodiments, the illderly-associated SNP is selected on the basis of its p value of association with illderly versus the wellderly, allele frequency and/or odds ratio in an illderly population.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. Table 1. illustrates Study populations and genotyping platforms

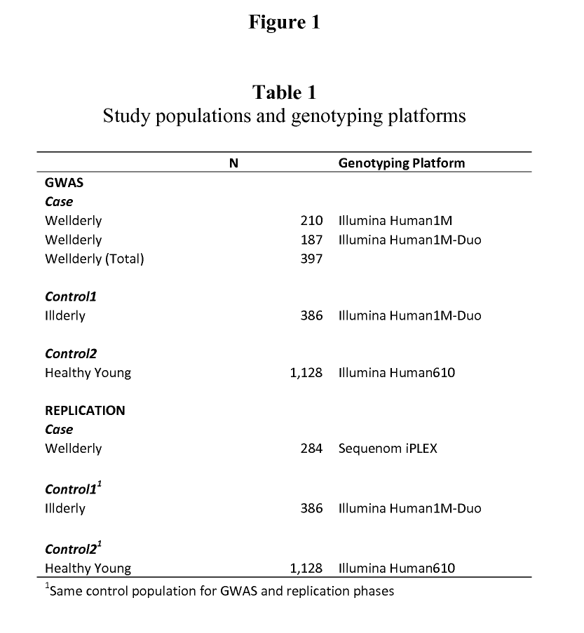

FIG. 2. Table 2. Illustrates Top 10 genomic regions in Wellderly-Illderly GWAS

FIG. 3. (Top) Table 3. Illustrates Top 10 genomic regions in Wellderly BMI GWAS; (Bottom) Table 4. Illustrates Three predicted functional non-synonymous SNPs in ANKDH1-EIF4EBP3

FIG. 4. Illustrates Wellderly-Illderly GWAS scatterplot of chromosomal position (x axis) versus—log10 GWAS P-value (Y axis).

FIG. 5. Illustrates Wellderly-Illderly EIF4EBP3 gene region scatterplot of chromosome position (x axis) versus—log10 P-value (left Y-axis), and recombination rate (right Y-axis). RefSeq genes are shown below chromosome 5 position on the X-axis. Scatterpoints are color coded with respect to LD-strength to most associated SNP rs250431. LD strength values shown are r2.

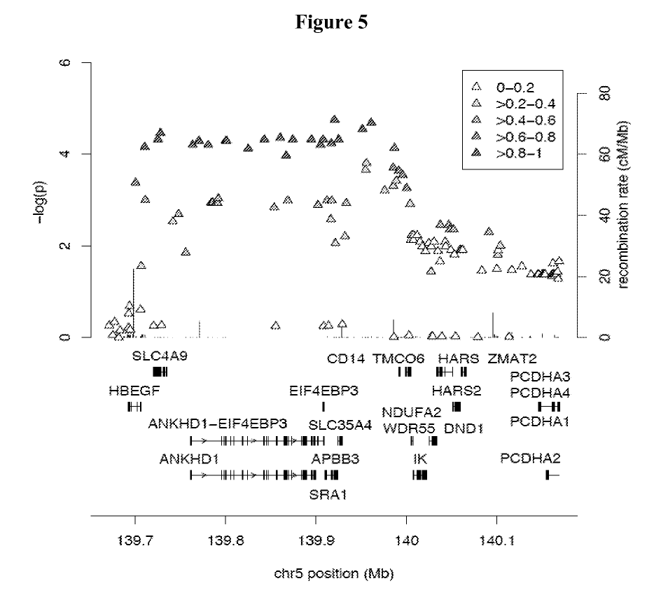

FIG. 6. Wellderly BMI scatterplot of chromosomal position (x axis) versus—log10 GWAS P-value (Y axis).

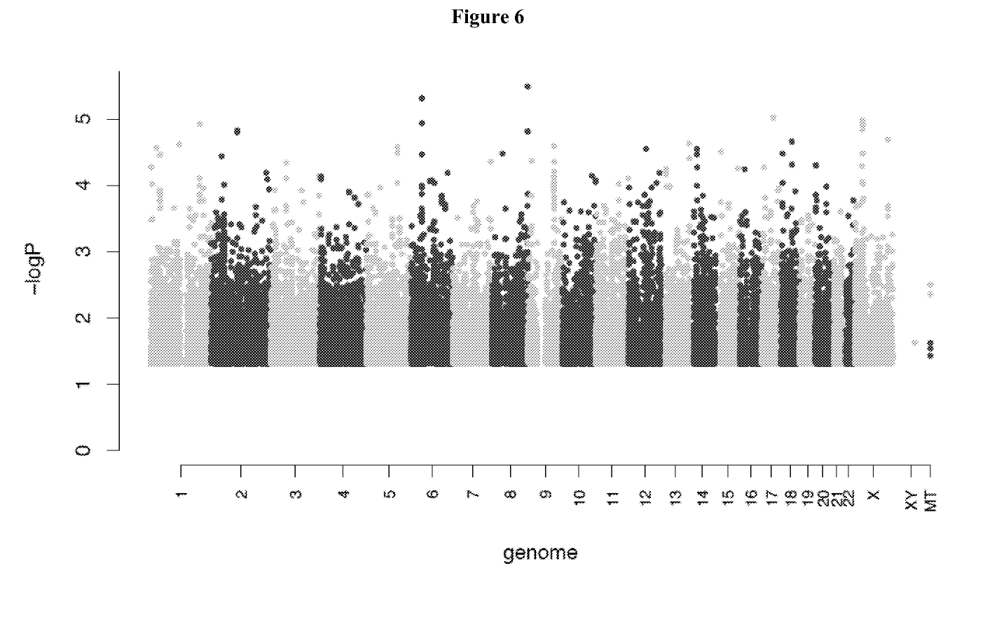

FIG. 7. Table 5.Illustrates Top 10 genomic regions for wellderly-young population control GWAS

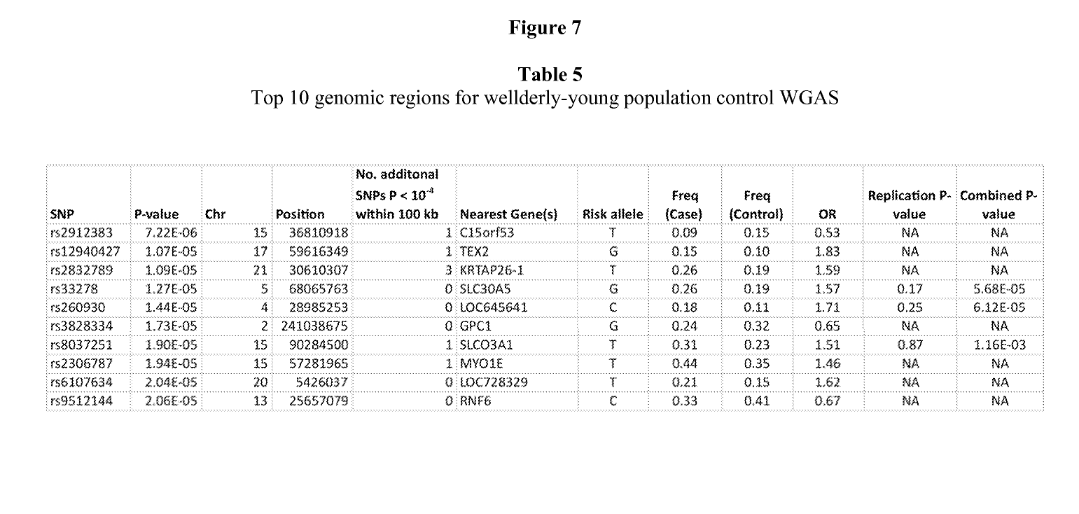

FIG. 8. Table 6.Illustrates Variables collected in the Wellderly cohort

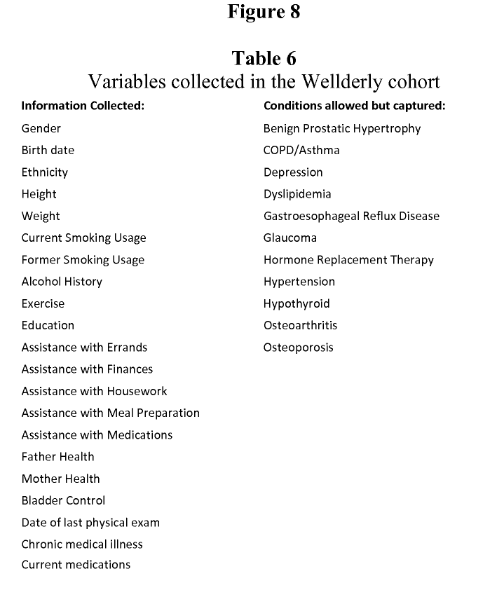

FIG. 9. (Top) Table 7.Illustrates Mean and median ages at enrollment (wellderly) and death (illderly); (Bottom) Table 8.Illustrates Genotype data quality metrics

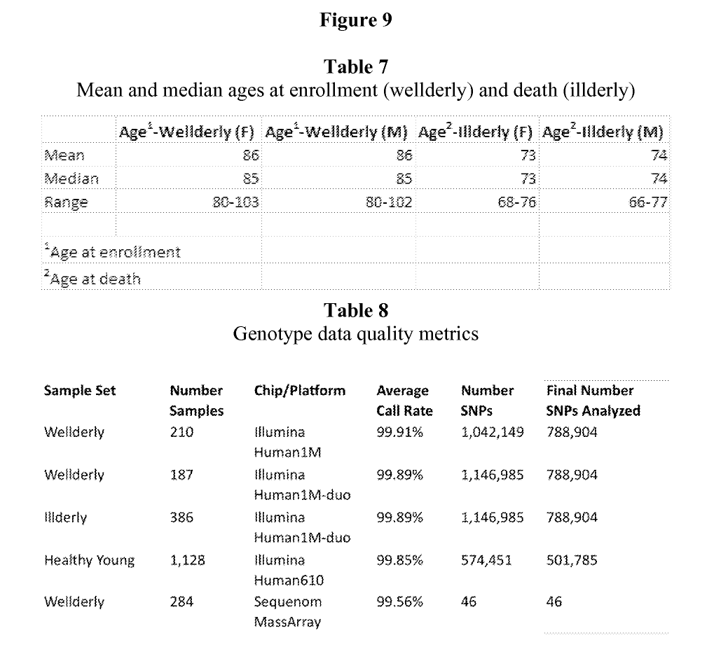

FIG. 10. Illustrates Wellderly-Illderly EIF4EBP3 gene region scatterplot of chromosome position (x axis) versus—log10 P-value (left Y-axis), and recombination rate (right Y-axis). RefSeq genes are shown below chromosome 5 position on the X-axis. Scatterpoints are color coded with respect to LD-strength to most associated SNP rs250431. LD strength values shown are r2. The haplotype associations are indicated as black lines from the start position of the first SNP to the position of the last SNP with y=−log(OMNIBUS P). P-values for SNPs in the region after adjusting for the most highly associated SNP rs250431 are shown as black filled circles.

FIG. 11. Illustrates Wellderly-Young population control GWAS scatterplot of chromosomal position (x axis) versus—log10 GWAS P-value (Y axis).

FIG. 12. Illustrates Histogram of wellderly and illderly sample, separated by gender, by birth year.

### DETAILED DESCRIPTION OF THE INVENTION

Genome-wide-association-studies (GWAS) have been applied to elucidate genes and pathways related to diseases. However, GWAS methodology has not been used to understand phenotype of healthy aging in humans. Described herein are certain genetic traits associated with the illderly and the wellderly. Certain genotypes are associated with unhealthy aging and/or onset of chronic age-related conditions including and not limited to cardiovascular disease, metabolic syndrome, and/or neurodegenerative disease. In elderly humans, a higher frequency of occurrence of certain SNPs and/or alleles is associated with unhealthy aging and/or decreased lifespan and/or chronic disease as cause of death.

Accordingly, described herein are methods for identification of individuals at risk for unhealthy aging and/or at risk of developing age-related fatal chronic diseases. Further described herein are certain SNPs associated with unhealthy aging and/or decreased lifespan in humans. Also provided herein are methods for modulation of the activity or expression of genomic regions in illderly- or wellderly-associated genomic regions and/or modulation of the level or activity of at least one expressed product thereof. Also described herein are methods for preventing unhealthy aging and/or delaying onset of age-related chronic conditions.

### Wellderly and Illderly Humans

As used herein, a wellderly human is of age 80 or greater with no significant chronic illnesses (exception mild hypertension) or long term use of medication. GWAS studies described herein also collected biographical information such as age, height, weight, ethnicity, family history, history of smoking, alcohol use, exercise, current medications, history of chronic medical illness, education, and any assistance requirements regarding general life functions (i.e., housework, medications, errands, finances, telephone use, meal preparation). Table 6 shows a complete list of variables collected and mild conditions that are permissible for this cohort.

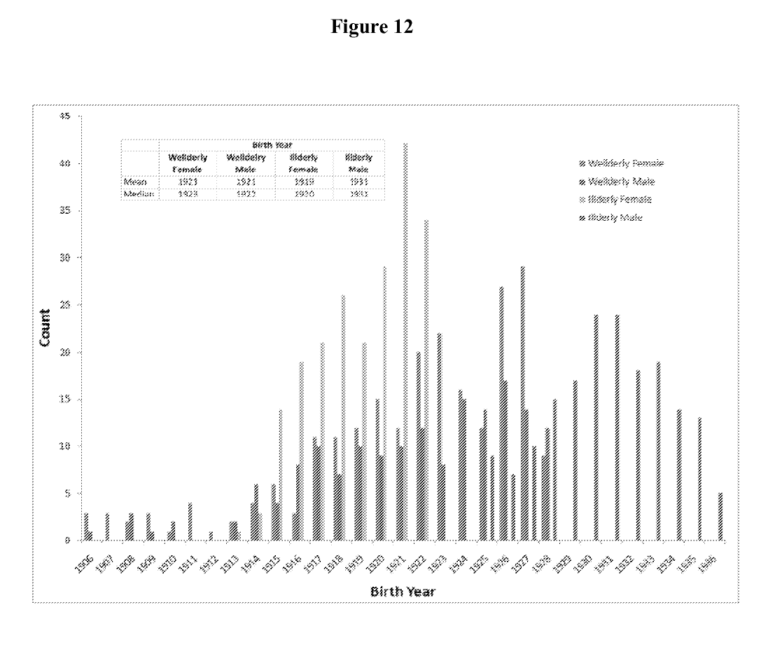

As used herein, illderly humans are humans of age 80 or greater who are either deceased due to chronic disease as cause of death, or are humans of age 80 or greater who suffer from a chronic and/or age-related disease including and not limited to cardiovascular disease, metabolic disease, and/or a neurodegenerative disease. For certain studies described herein, deceased members of two population-based cohorts were matched as close as possible for birth year and gender to the wellderly case group (FIG. 12). The Illderly control group all had chronic disease as cause of death. The MrOS cohort (The Osteoporotic Fractures in Men Study) is a prospectively collected cohort (without known osteoporosis) and includes 5995 men aged 65 years and older who were recruited primarily from population-based sources between March 2000 and April 2002 (22). The SOF cohort (Study of Osteoporotic Fractures) is a multi-center prospective observational study of a cohort of 10,366 women (without known osteoporosis) age 65 or older (23). For the controls derived from the MrOS cohort, the general causes of death are cancer (49%), cardiovascular disease/stroke (42%), dementia (6%), and chronic obstructive pulmonary disease (3%). For the SOF cohort, the general causes of death are cancer (52%), cardiovascular disease/stroke (27%), dementia (12%), and chronic obstructive pulmonary disease (9%). The mean age at death and range is shown in Table 7.

For certain genotyping studies described herein, Healthy Young Controls are part of a large population-based cohort of children of European ancestry with no known diseases. The mean age is 7 years (range=3 weeks-18 years).

### Genotyping

Genotyping is the process of determining the genotype of an individual by the use of biological assays. The genotyping methods described herein are for illustrative purposes only; any method of genotyping is optionally used to determine the genotype of an individual (e.g., whether wellderly or illderly).

In some instances, genotyping is carried out using polymerase chain reactions (PCR) using automated synthesizers. PCR analysis includes sequence-specific primers (SSP or allele specific primer extension [ASPE]) wherein specific variants are detected as positive or negative with the variant-specific primer; sequence-specific oligonucleotide probes (SSOP or allele-specific oligonucleotides [ASO]) wherein specific variants are detected via binding of the labeled-amplicon to the variant-specific probes on a dot blot; and/or restriction fragment length polymorphism analysis following PCR (PCR-RFLP), wherein variants are detected by visualization (e.g., fluorescent tags) of cut or uncut fragments following restriction endonuclease digestion of the PCR product. The location of the variant presents constraints on designing primers for SSP. SSOP and RFLP offer flexibility for primer design and primers can be anywhere, as long as they flank the variant. Often the basic PCR-based approaches are expensive and low throughput assays because the locus-specific primer is far from the variant of interest and/or genes that have paralogs require locus specific primers.

DNA sequencing is another technique used for genotyping. Large DNA fragments (e.g., chromosomes) are cut (with restriction enzymes) or sheared (with mechanical forces) into shorter DNA fragments. The fragmented DNA is cloned into a DNA vector, and amplified. Short DNA fragments purified from individual bacterial colonies are individually sequenced and assembled electronically into one long, contiguous sequence. Gaps in the assembled sequence are filled by primer walking High-throughput sequencing parallelizes the sequencing process, producing and sequencing very large numbers of sequences simultaneously. However, assembly of high throughput information is complex, particularly with sequence repeats often causing gaps in genome assembly.

Yet another technique for genotyping involves the use of allele-specific oligonucleotides (ASO). An ASO is typically an oligonucleotide of 15-21 nucleotide bases in length. It is specific for one version, or allele, of the DNA being tested. The length of the ASO, which strand it is chosen from, and the conditions by which it is bound to (and washed from) the target DNA all play a role in its specificity. An ASO probe can detect a difference of as little as 1 base (a single nucleotide polymorphism) in the target's genetic sequence. The ASO is labeled with a radioactive, enzymatic, or fluorescent tag that allows for detection after the ASO has bound to its target.

Another technique used for genotyping is a non-enzymatic method that involves hybridization to DNA microarrays. A single pool of DNA whose sequence is to be determined is fluorescently labeled and hybridized to a DNA microarray consisting of DNA probes that feature markers for genetic variations. Strong hybridization signals from a given spot on the array identifies its sequence in the DNA being sequenced.

Yet another technique for genotyping is the Illumina Methylation Assay technology. In this method, an ASO is used to detect one base pair difference (cytosine versus thymine) by measurement of methylation at a specific CpG site.

The Illumina whole-genome genotyping BeadChips technique involves hybridization of randomly fragmented genomic DNA to beads and is based on 3-micron silica beads that self assemble in microwells on substrates such as fiber optic bundles and/or planar silica slides. Each bead is covered with multiple copies of a specific oligonucleotide and/or an ASO that act as the capture sequences in genotyping assays. Attached DNA fragments are extended and/or bridge amplified. These templates are sequenced using sequencing-by-synthesis technology that employs reversible terminators with removable fluorescent dyes. High-sensitivity fluorescence detection is achieved using laser excitation and total internal reflection optics.

Sequence reads are aligned against a reference genome and genetic differences are called using data analysis software loaded on a Genome Analyzer. By way of example, Illumina's Human-1 BeadChip is used with a Genome Analyzer for identification of SNPs in the studies described herein in the figures and examples sections. By way of example, the SNP caller algorithm loaded on a Genome Analyzer is used for identification of SNPs in the studies described herein, and in the figures, tables and examples sections.

### Genome Wide Association Studies (GWAS)

A genome-wide association study (GWAS) is an approach that involves scanning markers across the complete sets of DNA, or genomes, of many people to find genetic variations associated with a particular disease. GWAS consist of screening the DNA of individuals with a large number (˜104→˜106) of genetic markers (SNPs) which span the entire genome. Significant “hits” (statistically significant deviations from an expected random inheritance of SNP alleles at a given locus if there were no association between the SNP and the disease/condition) are found based on a number of statistical tests. In specific embodiments, described herein are GWAS for identification of genetic variations associated with the phenotype of healthy aging.

A GWAS described herein is carried out using two groups of individuals, a cohort with an age-related disease and a cohort associated with a phenotype of healthy aging (i.e., free of an age-related disease). DNA from each individual in both cohorts is genotyped as described above. The techniques described above allow for identification of markers of genetic variation (single nucleotide polymorphisms, or SNPs). Where certain genetic variations are found to be statistically significantly more frequent in people with the disease compared to people without disease, the variations are said to be “associated” with the disease.

In some embodiments, the associated variants themselves do not directly cause the disease; the variants “tag along” with the actual causal variants. Accordingly, also contemplated within the scope of embodiments described herein are any additional steps, such as deep sequencing of DNA base pairs in that particular region of the genome, that identify the exact causal variant involved in the disease. Also contemplated within the scope of embodiments described herein is modulation of any causal variants associated with healthy aging and/or preventing or delaying onset of age-related diseases.

Thus, in one embodiment, described herein is the following study: Using a 1 million single nucleotide polymorphism (SNP) array, DNA samples from individuals who were at least age 80 and had no chronic disease were compared with individuals who were deceased from chronic disease, matched for birth year, gender and ancestry. The top SNPs results were assessed in a second healthy aging cohort for replication. Multiple SNPs in the translator regulator EIF4EBP3, a key gene in the mTOR pathway, were significantly associated with healthy aging.

A hypothesis-free approach was used to analyze the phenotype of healthy aging. Multiple sequence variants in the gene EIF4EBP3 were identified to be associated and replicated via targeted genotyping of a second cohort. The association was noted in comparison with individuals who had died from chronic diseases, but born in approximately the same birth year, to attempt to match for environmental exposure.

The analysis yielded significant association with EIF4EBP3, as described herein in the figures, tables and Examples sections. EIF4EBP3 along with EIF4EBP1 and EIF4EBP2 produce proteins that bind to eIF4E which prevent eIF4G binding thus inhibiting translation.

Along with ribosomal protein S6 (S6K1), 4E-BP is one of the two most important translational regulators in the mTOR pathway which is responsible for linking nutrient availability and the control of cell growth and metabolism. In a mouse model, S6K1−/− mice had extended lifespan (particularly among females), less insulin resistance, improved glucose homeostasis, and improved immune and motor function and bone volume compared with wild type mice indicating that S6K1 plays a role in extending lifespan in rodents.

### Single Nucleotide Polymorphisms

A single nucleotide polymorphism (SNP) is a DNA sequence variation wherein there is a single nucleotide difference in the genome and/or other shared sequence between members of a species. In some embodiments, described herein are certain SNPs that are associated with healthy aging. In some embodiments, certain SNPs are associated with the illderly and the presence of such SNPs indicates that an individual has a higher risk of developing certain age-related conditions described herein. These are referred herein as “illderly SNPs.” In some embodiments, certain SNPs are associated with the wellderly and the presence of such SNPs indicates that an individual has a higher probability of healthy aging, i.e., a lower risk of developing age-related diseases or conditions. These are referred herein as “wellderly SNPs.” Disclosed herein are methods for identifying whether or not an individual has an illderly SNP or a wellderly SNP, and the use of such information to provide the individual with appropriate medical advice (including, e.g., the therapeutic interventions described herein).

In some embodiments, SNPs associated with age-related diseases include and are not limited to SNPs in the genomic region spanning from position 139710628 to 139986614 base pairs on human chromosome 5 based on hg19, GRCh37 Genome Reference Consortium Human Reference 37. In some of such embodiments, SNPs associated with age-related diseases include and are not limited to rs1862176, rs6879217, rs34507975, rs6860077, rsl 1168051, rs17286676, rs13171154, rs35309382, rs6872579, rs7356554, rs975243, rs1432959, rs12717986, rs10042299, rs12717988, rs7715360, rs7701365, rs3822328, rs10476907, rs1019933, rs10041762, rs9687753, rs12109242, rs6876865, rs6871703, rs7712617, rs6579768, rs3733681, rs9324644, rs250432, rs250424, rs250425, rs801460, rs801459, rs10463297, rs250426, rs2249213, rs250431, rs250430, rs250429, rs2253378, rs4912715, rs801458, rs4279384, rs1864255, rs2569163, rs1835148, rs7444290, rs778583, rs778584, rs7721577, and rs778587.

In certain specific embodiments, SNPs associated with age-related diseases include and are not limited to rs250431, rs2071004, rs1863291, rs7609512, rs7016232, rs10758570, rs10866584, rs17620029, rs4765461, rs6903798, rs1386267, rs5971801, rs3749898, rs11590076, rs5927244, rs2889450, rs7815797, rs1929363, rs8095607, rs755993, rs1051309, rs3172957, and rs3752704.

### Definitions

An “age-related disease or condition” refers to a disease with an onset mainly in middle age or later. Non-limited examples include metabolic disorders (e.g., diabetes, including Type II diabetes), neurodegenerative diseases (e.g., Alzheimer's disease, dementia, mild cognitive impairment, Parkinson's disease, stroke), bone-loss disorders (e.g., osteoporosis), degenerative arthritis, heart diseases and destructive eye diseases. Examples of diseases associated with the elderly include, and are not limited to, pre-diabetes, diabetes (including Type II diabetes), obesity, osteoporosis, coronary artery disease, cerebrovascular disease, heart attack, stroke, peripheral arterial disease, aortic valve disease, stroke, mild cognitive impairment, pre-dementia, dementia, macular degeneration, and cataracts.

A “genomic region” is a segment of DNA that comprises one or more genes and/or genetic elements including and not limited to introns, exons, chromosomal DNA, non-chromosomal DNA, plasmids, transposons, autosomes, sex chromosomes or the like. A genomic region comprises both functional and/or non-functional genes and/or genetic elements.

An “allele” is one of the variant forms of a gene or nucleotide sequence at a particular locus on a chromosome. “Allele frequency” is the proportion of all copies of a gene that is made up of a particular gene variant (allele). With respect to SNPs associated with diseases or conditions, one allele is often referred to as the “risk” allele which means this is the variant form of the SNP most often found associated with that particular disease or condition. Association in this context means that one of the SNP alleles (the “risk” allele) is found at a higher frequency in cases when compared to controls. Since most SNPs are biallelic (a SNP which detects two variant forms) one form is designated the risk allele and the other form is designated the non-risk allele. Herein, the “risk allele” refers to the allele associated with the illderly and the “non-risk allele” refers to the allele associated with the wellderly.

As used herein, the term “inhibitor” refers to a molecule which is capable of inhibiting (including partially inhibiting or allosteric inhibition) one or more of the biological activities of a target molecule, e.g., mTOR kinase, S6K1, 4E-BP3, or the like. Inhibitors act, for example, by reducing or suppressing the activity of a target molecule and/or reducing or suppressing signal transduction. In some embodiments, an inhibitor described herein causes substantially complete inhibition of the target molecule (e.g., mTOR kinase, S6K1, 4E-BP3, or the like). In some embodiments, an inhibitor is a partial inhibitor. The phrase “partial inhibitor” refers to a molecule which can induce a partial response for example, by partially reducing or suppressing the activity of a target molecule and/or partially reducing or suppressing signal transduction. In some instances, a partial inhibitor mimics the spatial arrangement, electronic properties, or some other physicochemical and/or biological property of the inhibitor. In some instances, in the presence of elevated levels of an inhibitor, a partial inhibitor competes with the inhibitor for occupancy of the target molecule and provides a reduction in efficacy, relative to the inhibitor alone. In some embodiments, an inhibitor described herein is an allosteric modulator of a target molecule (e.g., mTOR kinase, S6K1, 4E-BP3, or the like). As used herein, “substantially complete inhibition” means, for example, >95% inhibition of one or more targeted molecules (e.g., mTOR kinase, S6K1, 4E-BP3, or the like). In other embodiments, “substantially complete inhibition” means, for example, >90% inhibition of one or more targeted molecules (e.g., mTOR kinase, S6K1, 4E-BP3, or the like). In some other embodiments, “substantially complete inhibition” means, for example, >80% inhibition of one or more targeted molecules (e.g., mTOR kinase, S6K1, 4E-BP3, or the like). As used herein, “partial inhibition” means, for example, between about 40% to about 60% inhibition of one or more targeted molecules (e.g., mTOR kinase, S6K1, 4E-BP3, or the like). In other embodiments, “partial inhibition” means, for example, between about 50% to about 70% inhibition of one or more targeted molecules (e.g., mTOR kinase, S6K1, 4E-BP3, or the like).

As used herein the term “treatment”, “treat”, or “treating” includes achieving a therapeutic benefit and/or a prophylactic benefit. Therapeutic benefit is meant to include eradication or amelioration of the underlying disorder or condition being treated (e.g., an age-related disease or condition described herein). For example, therapeutic benefit includes alleviation or partial and/or complete halting of progression of the disease and/or delaying onset of disease. Also, a therapeutic benefit is achieved with the eradication or amelioration of one or more of the physiological or psychological symptoms associated with the underlying condition such that an improvement is observed in the patient, notwithstanding the fact that the patient is still affected by the condition. For example, in an individual suffering from Alzheimer's disease, therapeutic benefit includes alleviation or partial and/or complete halting of disease progression as indicated by progressive loss of bodily functions. A prophylactic benefit of treatment includes prevention of a condition, retarding the progress of a condition, or decreasing the likelihood of occurrence of a condition. As used herein, “treat”, “treating” or “treatment” includes prophylaxis.

### Genomic Region

Described herein are studies that identify a high degree of association of certain genomic regions with age-related conditions. Also described herein are methods for modulating the activity and/or expression of at least one gene in the genomic region associated with age-related disease and/or modulating the level or activity of at least one expressed product thereof. Also described herein are methods for modulating translation activity in a human.

In one embodiment, a genomic region associated with unhealthy aging is the 4EIFBP3 region. As used herein, the 4EIFBP3 genomic region refers to the genomic region spanning 137,907,435-141,909,347 by on human chromosome 5, and/or paralogs thereof.

In some embodiments, genetic elements within the EIF4EBP3 genomic region that comprise SNPs associated with age-related diseases and/or unhealthy aging include the region encompassing SLC4A9 to SLC35A4 (spanning 139,719,971-139,928,867 by on human chromosome 5), the region encompassing the gene APPB3 (spanning 139,918,037-139,924,373 by on human chromosome 5), the region encompassing the gene ANKHD1-EIF4EBP3 (spanning 139,761,613-139,909,347 on human chromosome 5), the region encompassing the gene EIF4BP3 (139,907,435-139,909,347 by on human chromosome 5), the region encompassing the gene ANKHD1 (139,761,613-139,929,163 by on human chromosome 5), the region encompassing the gene SLC4A9 (139,719,971-139,734,906 by on human chromosome 5), the region encompassing the gene SLC35A4 (139,924,604-139,928,867 by on human chromosome 5), and/or the region encompassing the gene SRA1 (139,909,837-139,917,862 by on human chromosome 5).

In specific embodiments, the EIF4EBP3 genomic region encompassing the EIF4EBP3 gene (located at 139,907,435-139,909,347 by on human chromosome 5) comprises SNPs associated with age-related diseases and/or unhealthy aging and such association is statistically significant. In some embodiments, genes in the 4EIFBP3 genomic region include and are not limited to SLC4A9, ANKHD1, ANKHD1-EIF4EBP3, EIF4EPB3, SRA1, APPB3, and SLC35A4 and comprise SNPs associated with age-related diseases and/or unhealthy aging.

Accordingly, provided herein are methods of promoting healthy aging in a human or preventing a disease associated with the elderly in a human comprising administering to the human an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof.

In some of such embodiments, provided herein are methods for promoting healthy aging in a human or preventing a disease associated with the elderly in a human comprising administering to the human an agent that (a) modulates the activity or expression of the 4EIFBP3 gene in an illderly- or wellderly-associated genomic region spanning from 139,907,435-139,909,347 by on human chromosome 5, based on hg19, GRCh37 Genome Reference Consortium Human Reference 37, or (b) modulates the level or activity of at least one expressed product thereof. By way of example, an expressed product of the 4EIFBP3 genomic region and/or the 4EIFBP3 gene is the protein 4E-BP3.

In some of such embodiments, provided herein are methods for promoting healthy aging in a human or preventing a disease associated with the elderly in a human comprising administering to the human an agent that (a) modulates the activity or expression of one or more of SLC4A9, ANKHD1, ANKHD1-EIF4EBP3, EIF4EPB3, SRA1, APPB3, and/or SLC35A4 genes in an illderly- or wellderly-associated genomic region spanning from 139,907,435-139,909,347 by on human chromosome 5, based on hg19, GRCh37 Genome Reference Consortium Human Reference 37, or (b) modulates the level or activity of at least one expressed product thereof.

### 4EIFBP1 Genomic Region

In another embodiment, a genomic region associated with unhealthy aging is the 4EIFBP1 region. As used herein, the 4EIFBP1 genomic region refers to the genomic region spanning 36,007,177-40,037,040 by on human chromosome 8, and/or paralogs thereof. In some embodiments, the 4EIFBP1 genomic region encompasses the 4EIFBP1 gene (spanning 38,007,177-38,037,040 by on human chromosome 8).

Accordingly, provided herein are methods of promoting healthy aging in a human or preventing a disease associated with the elderly in a human comprising administering to the human an agent that (a) modulates the activity or expression of at least one gene in an illderly- or wellderly-associated genomic region spanning from position 36,007,177-40,037,040 by on human chromosome 8, based on hg19, GRCh37 Genome Reference Consortium Human Reference 37, or (b) modulates the level or activity of at least one expressed product thereof.

In some of such embodiments, provided herein are methods for promoting healthy aging in a human or preventing a disease associated with the elderly in a human comprising administering to the human an agent that (a) modulates the activity or expression of the 4EIFBP1 gene in an illderly- or wellderly-associated genomic region spanning from 38,007,177-38,037,040 by on human chromosome 8, based on hg19, GRCh37 Genome Reference Consortium Human Reference 37, or (b) modulates the level or activity of at least one expressed product thereof.

### 4EIFBP2 Genomic Region

In another embodiment, a genomic region associated with unhealthy aging is the 4EIFBP2 region. As used herein, the 4EIFBP2 genomic region refers to the genomic region spanning 69,833,928-73,853,676 by on human chromosome 10, and/or paralogs thereof. In some embodiments, the 4EIFBP2 genomic region encompasses the 4EIFBP2 gene (spanning 71,833,928-71,853,676 by on human chromosome 10).

Accordingly, provided herein are methods for promoting healthy aging in a human or preventing a disease associated with the elderly in a human comprising administering to the human an agent that (a) modulates the activity or expression of at least one gene in an illderly- or wellderly-associated genomic region spanning from position 69,833,928-73,853,676 by on human chromosome 10, based on hg19, GRCh37 Genome Reference Consortium Human Reference 37, or (b) modulates the level or activity of at least one expressed product thereof.

In some of such embodiments, provided herein are methods for promoting healthy aging in a human or preventing a disease associated with the elderly in a human comprising administering to the human an agent that (a) modulates the activity or expression of the 4EIFBP2 gene in an illderly- or wellderly-associated genomic region spanning from 71,833,928-71,853,676 by on human chromosome 10, based on hg19, GRCh37 Genome Reference Consortium Human Reference 37, or (b) modulates the level or activity of at least one expressed product thereof.

Genomic Region Products

RNA

In some embodiments, the methods for promoting healthy aging in a human or preventing a disease associated with the elderly in a human comprise administering to the human an agent that modulates the activity of an RNA that is an expressed transcribed product of the genomic region. In some of such embodiments, the RNA is an mRNA and/or a tRNA that is involved in translation. Accordingly, in some embodiments, the methods for promoting healthy aging in a human or preventing a disease associated with the elderly in a human comprise administering to the human an agent that reduces or prevents RNA synthesis including and not limited to an RNAi.

Proteins

In some embodiments, the methods for promoting healthy aging in a human or preventing a disease associated with the elderly in a human comprise administering to the human an agent that modulates the activity of a protein that is an expressed product of the genomic region. In some embodiments, an expressed protein product of the 4EIFBP1, 4EIFBP2 or 4EIFBP3 genomic regions is a translation inhibitor protein 4E-BP.

In some embodiments, the expressed protein product of the 4EIFBP3 genomic region is a product of the 4EIFBP3 gene. In some embodiments, the expressed protein product of the 4EIFBP3 genomic region is a translation inhibitor protein 4E-BP. In some embodiments, the expressed protein product of the 4EIFBP3 genomic region is the translation inhibitor protein 4E-BP3.

In some embodiments, the expressed protein product of the 4EIFBP3 genomic region is a product of the 4EIFBP1 gene. In some embodiments, the expressed protein product of the 4EIFBP1 genomic region is a translation inhibitor protein 4E-BP. In some embodiments, the expressed protein product of the 4EIFBP 1 genomic region is the translation inhibitor protein 4E-BP1.

In some embodiments, the expressed protein product of the 4EIFBP3 genomic region is a product of the 4EIFBP2 gene. In some embodiments, the expressed protein product of the 4EIFBP2 genomic region is a translation inhibitor protein 4E-BP. In some embodiments, the expressed protein product of the 4EIFBP2 genomic region is the translation inhibitor protein 4E-BP2.

Also contemplated within the scope of the embodiments described herein are any other translation inhibitor proteins (4E-BPs) and/or any other protein product of the genomic region 4EIFBP3. Also contemplated within the scope of the embodiments described herein are any other translation inhibitor proteins (4E-BPs) and/or any other protein product of the genomic region 4EIFBP2. Also contemplated within the scope of the embodiments described herein are any other translation inhibitor proteins (4E-BPs) and/or any other protein product of the genomic region 4EIFBP 1.

### 4E-BPs

4E-BPs are translation inhibitory proteins that bind to eukaryotic initiation factors and prevent initiation of translation and/or formation of translation initiation complexes. Eukaryotic initiation factors such as eIF-4E and eIF-4G, and the S6 ribosomal protein are involved in translational regulation. Several stimuli, including growth factors and cytokines, regulate the eIF-4F initiation complex and the S6 ribosomal protein by initiating a phosphorylation cascade involving the sequential activation of PI3-K, PDK1/2, Akt/PKB, and FRAP/mTOR kinase. In some instances, kinases including, and not limited to, FRAP/mTOR kinase phosphorylate a 4E-BP (e.g., 4E-BP3), leading to its dissociation from eIF-4E. Dissociation of a 4E-BP from eIF-4E activates eIF-4E. Activation of eIF-4E triggers association of eIF-4E to eIF-4G. The binding of eIF-4E to eIF-4G results in an active eIF-4F initiation complex that then initiates translation.

In some instances, translation is initiated by an alternative pathway wherein MNK1/2 (activated by ERK and p38 MAPK) phosphorylates and activates eIF-4E with subsequent formation of the active eIF-4F complex that initiates translation. In some instances, activation of mTOR activates p70S6 Kinase (S6K) which then phosphorylates the S6 ribosomal protein. Phosphorylation of S6 ribosomal protein induces protein synthesis at the ribosome.

Excess phosphorylation of a 4E-BP (e.g., hyperphosphorylation of a 4E-BP) causes dissociation of the 4E-BP from eIF-4E, which allows the eIF-4E to form the initiation complex eIF-4F. As used herein, “hyperphosphorylation of a 4E-BP” means that the 4E-BP phosphorylation level is such that the 4E-BP would dissociate from its binding partner (e.g., eIF-4E), thereby allowing for initiation of translation.

In some embodiments of the methods for promoting healthy aging described herein, modulation of binding of 4E-BP to binding partners (e.g., 4EIFE) reduces or suppresses 4E-BP mediated translation. In some embodiments of the methods for promoting healthy aging described herein, modulation of 4E-BP binding and/or 4E-BP mediated translation is effected by modulation of phosphorylation levels of 4E-BPs. Accordingly, in some embodiments of the methods for promoting healthy aging described herein, modulation of phosphorylation levels of 4E-BPs (e.g., by increasing or decreasing phosphorylation) of a 4E-BP allows for modulation of translation. A decrease in phosphorylation levels of a 4E-BP (e.g., by inhibition or partial inhibition of an upstream kinase that phosphorylates 4E-BP, such as mTOR, AKT or the like) allows for association of 4E-BP with eIF-4E, thereby preventing subsequent formation of the initiation complex and reducing translation.

Accordingly, provided herein, in some embodiments, are methods for promoting healthy aging in a human or preventing a disease associated with the elderly in a human comprising administering to the human an agent that modulates the activity of a 4E-BP so as to prevent translation associated with 4E-BP binding to eukaryotic initiation factors.

In some embodiments, an agent for promoting healthy aging in a human or preventing a disease associated with the elderly in a human reduces or prevents hyperphosphorylation of a 4E-BP (i.e., 4E-BP is activated), thereby preventing dissociation of 4E-BP from eIF4E. The 4E-BP-eIF4E complex reduces or suppresses translation by (a) slowing down and/or hindering the association of eIF-4E to eIF-4G and/or (b) preventing or hindering formation of an active eIF-4F complex that initiates translation.

In some embodiments, an agent for promoting healthy aging in a human or preventing a disease associated with the elderly in a human reduces or prevents hyperphosphorylation of a 4E-BP by inhibiting or partially inhibiting the activity of a kinase (e.g., any upstream kinase described above) that phosphorylates a 4E-BP. In some embodiments, an agent that reduces or prevents hyperphosphorylation of a 4E-BP inhibits or partially inhibits the activity of any kinase in the mTOR pathway.

In some embodiments, an agent for promoting healthy aging in a human or preventing a disease associated with the elderly mimics the 4E-BP hypophosphorylated state and competes with eIF4G in binding to eIF4E to form eIF4F.

In some other embodiments, an agent for promoting healthy aging in a human or preventing a disease associated with the elderly increases or promotes binding of a eukaryotic translation initiation factor 4E-binding protein (4E-BP) to eukaryotic translation initiation factor 4E (eIF4E), thereby preventing translation of the associated genes.

In some embodiments, an agent for promoting healthy aging in a human or preventing a disease associated with the elderly in a human disrupts binding of eIF4E to eIF4G to decrease cap-dependent translation and/or increases nuclear sequestration of eIF4E. In some embodiments, an agent for promoting healthy aging in a human or preventing a disease associated with the elderly in a human reduces translation activity in a human.

In specific embodiments, provided herein are methods for promoting healthy aging in a human or preventing a disease associated with the elderly in a human comprising administering to the human an agent that modulates the activity of 4E-BP3 so as to prevent translation associated with 4E-BP binding to eukaryotic initiation factors.

In some embodiments, an agent for promoting healthy aging in a human or preventing a disease associated with the elderly in a human reduces or prevents hyperphosphorylation of 4E-BP3 (i.e., 4E-BP3 is activated), thereby preventing dissociation of 4E-BP3 from eIF4E. The 4E-BP3-eIF4E complex reduces or suppresses translation by (a) slowing down and/or hindering the association of eIF-4E to eIF-4G and/or (b) preventing or hindering formation of an active eIF-4F complex that initiates translation.

In some embodiments, an agent for promoting healthy aging in a human or preventing a disease associated with the elderly in a human reduces or prevents hyperphosphorylation of 4E-BP3 by inhibiting or partially inhibiting the activity of a kinase (e.g., any upstream kinase described above) that phosphorylates 4E-BP3. In some embodiments, an agent that reduces or prevents hyperphosphorylation of 4E-BP3 inhibits or partially inhibits the activity of any kinase in the mTOR pathway.

In some embodiments, an agent for promoting healthy aging in a human or preventing a disease associated with the elderly mimics the 4E-BP3 hypophosphorylated state and competes with eIF4G in binding to eIF4E to form eIF4F.

In some other embodiments, an agent for promoting healthy aging in a human or preventing a disease associated with the elderly increases or promotes binding of 4E-BP3 to eukaryotic translation intitiation factor 4E (eIF4E).

In some embodiments, a 4E-BP3 modulator disrupts binding of eIF4E to eIF4G to decrease cap-dependent translation and/or increases nuclear sequestration of eIF4E. In some embodiments, a 4E-BP3 modulator reduces translation activity in a human. 4E-BP modulators

As used herein, a 4E-BP modulator is an agent that modulates (e.g., increases or decreases) translational activity in an individual. In some embodiments, such translation activity is associated with cell growth, metabolism and/or mitochondrial energy and/or lifespan pathways.

In specific embodiments, a 4E-BP modulator is a 4E-BP blocker. As used herein, a “4E-BP blocker” reduces or suppresses hyperphosphorylation of a 4E-BP, changes hyperphosphorylated 4E-BP to hypophosphorylated 4E-BP, slows down and/or hinders the association of eIF-4E to eIF-4G, prevents or hinders formation of an active eIF-4F complex that initiates translation, inhibits or partially inhibits the activity of a kinase (e.g., any upstream kinase such as mTOR, S6 kinase, or the like as described above) that phosphorylates a 4E-BP, increases or promotes binding of a eukaryotic translation initiation factor 4E-binding protein (4E-BP) to eukaryotic translation intitiation factor 4E (eIF4E), disrupts binding of eIF4E to eIF4G to decrease cap-dependent translation and/or increases nuclear sequestration of eIF4E, and/or mimics the 4E-BP hypophosphorylated state and competes with eIF4G in binding to eIF4E to form eIF4F initiation complex, or any combination thereof. As used herein, a 4E-BP blocker reduces or suppresses translation of any proteins for which translation is mediated by 4E-BP activity (also referred herein as 4E-BP mediated translation).

In specific embodiments, a 4E-BP blocker is a 4E-BP3 blocker.

Accordingly, provided herein are methods for promoting healthy aging in a human or preventing a disease associated with the elderly in a human comprising administering to the human an agent that modulates the activity and/or binding of 4E-BP3 in an illderly- or wellderly-individual. In some embodiments, an agent that modulates the activity and/or binding of 4E-BP3 in an illderly- or wellderly-individual is a 4E-BP3 blocker. In some embodiments, a 4E-BP3 blocker reduces or inhibits 4E-BP3 mediated translation.

Examples of 4E-BP blockers include and are not limited to compounds of Formula I:

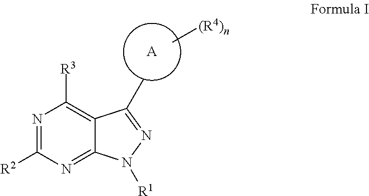

wherein:
ring A is a substituted or unsubstituted monocyclic or bicyclic aryl, or monocyclic or bicyclic heteroaryl;
R¹ is H, substituted substituted or unsubstituted alkyl, substituted or unsubstituted heteroalkyl, substituted or unsubstituted cycloalkyl, substituted or unsubstituted heterocycloalkyl, substituted or unsubstituted alkylcycloalkyl, substituted or unsubstituted alkylheterocycloalkyl, —C(=O)R⁵, —C(═O)OR⁵, —C(═O)NHR⁵, —S(═O)R⁵, or —S(═O)₂R⁵;
each R⁵ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₁₀cycloalkyl, substituted or unsubstituted C₂-C₁₀heterocycloalkyl, substituted or unsubstituted aryl, substituted or unsubstituted heteroaryl, —C₁-C₂alkylene-(substituted or unsubstituted C₃-C₁₀cycloalkyl), —C₁-C₂alkylene-(substituted or unsubstituted C₂-C ₁₀heterocycloalkyl), —C₁-C₂alkylene-(substituted or unsubstituted aryl), and —C₁-C₂alkylene-(substituted or unsubstituted heteroaryl);
R² is H, halogen, OH, —CN, —OR⁶, —SR⁶, —NH₂, —NHR⁶, —N(R⁵)₂,
each R⁶ is independently selected from H, —C(═O)R⁵, —C(═O)OR⁵, —C(═O)NHR⁵, substituted or unsubstituted C₁-C₆alkyl, substituted or unsubstituted C₁-C₆heteroalkyl, substituted or unsubstituted C₁-C₆fluoroalkyl, substituted or unsubstituted C₃-C₁₀cycloalkyl, substituted or unsubstituted C₂-C ₁₀heterocycloalkyl, substituted or unsubstituted aryl, substituted or unsubstituted heteroaryl, —C₁-C₂alkylene-(substituted or unsubstituted C₃-C₁₀cycloalkyl), —C₁-C₂alkylene-(substituted or unsubstituted C₂-C₁₀heterocycloalkyl), —C₁-C₂alkylene-(substitut or unsubstituted aryl), and —C₁-C₂alkylene-(substituted or unsubstituted heteroaryl);
R³ is H, halogen, —OH, —CN, —OR⁶, —SR⁶, —NH₂, —NHR⁶, —N(R⁵)₂, —NHC(═O)R⁵, —C(50 O)R⁵, —C(═O)OR⁵, —C(═O)NHR⁵, —S(═O)R⁵, —S(═O)₂R⁵, —S(═O)₂NHR⁵;
each R⁴ is independently selected from H, halogen, —CN, —OH, —OR⁶, —SR⁶, —S(═O)R⁵, —S(═O)₂R⁵, —NHS(═O)₂R⁵, substituted or unsubstituted C₁-C₆alkyl, substituted or unsubstituted C₁-C₆fluoroalkyl, substituted or unsubstituted C₁-C₆fluoroalkoxy, substituted or unsubstituted C₁-C₆alkoxy, and substituted or unsubstituted C₁-C₆heteroalkyl; and
n is 0, 1, 2, 3, or 4.

In some embodiments of Formula I, ring A is selected from phenyl, naphthyl, indanyl, quinolinyl, indolyl, indazolyl, benzoxazolyl or benzimidazolyl.

In some embodiments, a compound of Formula I has the structure of Formula II:

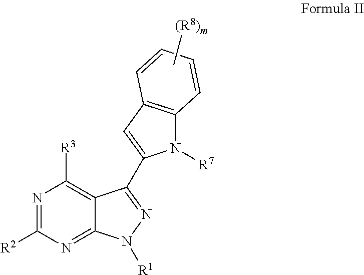

wherein:
R⁷ is H, substituted or unsubstituted C₁-C₆alkyl, —C(═O)R⁵, —S(═O)R⁵, or —S(═O)₂R⁵;
each R⁸ is independently selected from H, halogen, —CN, —OH, —OR⁶, —SR⁶, —S(═O)R⁵, —S(═O)₂R⁵, —NHS(═O)₂R⁵, substituted or unsubstituted C₁-C₆alkyl, substituted or unsubstituted C₁-C₆fluoroalkyl, substituted or unsubstituted C₁-C₆fluoroalkoxy, substituted or unsubstituted C₁-C₆alkoxy, and substituted or unsubstituted C₁-C₆heteroalkyl; and
m is 0, 1, 2, 3, or 4.

In some embodiments, a compound of Formula II has a structure of Formula III:

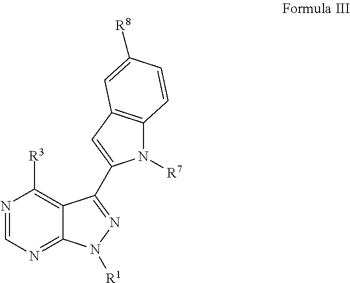

wherein
R¹ is H, or substituted substituted or unsubstituted C₁-C₆alkyl, substituted or unsubstituted cycloalkyl, or substituted or unsubstituted alkylcycloalkyl;
R³ is H, halogen, —OH, —CN, —OR⁶, —NH₂, or —NHR⁶;
R⁷ is H, or substituted or unsubstituted C₁-C₆alkyl; and
R⁸ is H, halogen, —CN, —OH, or —OR⁶.

In some embodiments, a compound of Formula I, or Formula II or Formula III is a compound wherein R¹ is H, methyl, ethyl, propyl, isopropyl, butyl, isobutyl, neopentyl, cyclopropyl, cyclopropylmethyl, cyclobutyl, cyclobutylmethyl, or acetyl. In some embodiments, a compound of Formula I, or Formula II or Formula III is a compound wherein R⁷ is H, methyl, ethyl, propyl, isopropyl, butyl, isobutyl, neopentyl, cyclopropyl, cyclopropylmethyl, cyclobutyl, cyclobutylmethyl, or acetyl. In some embodiments, a compound of Formula I, or Formula II or Formula III is a compound wherein R³ is H, halogen, —OH, —CN, —NH₂, —NHR⁶, —N(R⁵)₂, or —NHC(═O)R⁵. In specific embodiments, a compound of Formula I, or Formula II or Formula III is

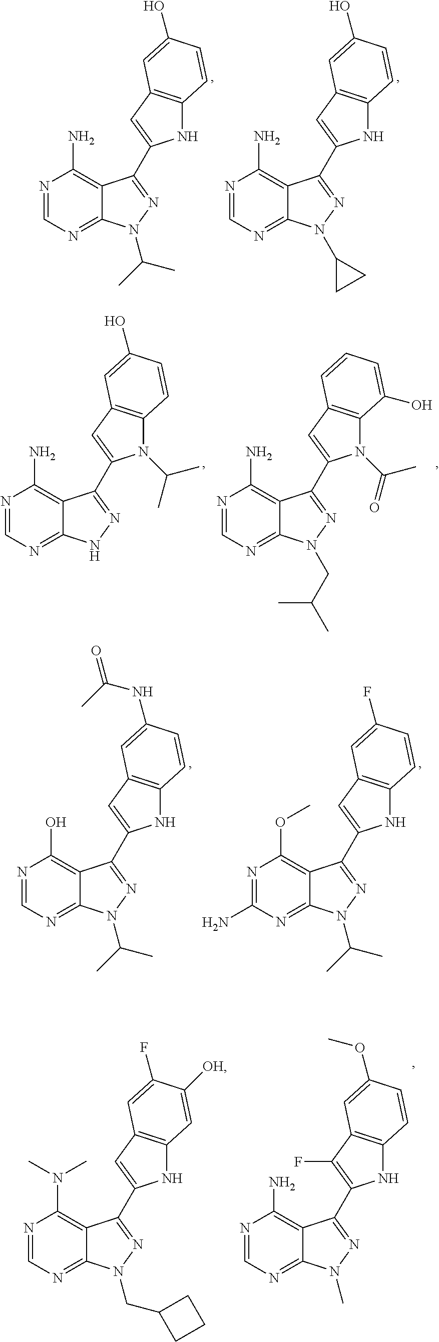

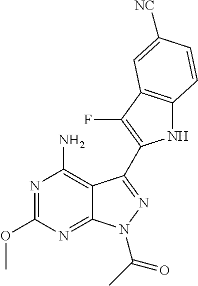

or the like.

In some embodiments, compounds of Formula I or Formula II or Formula III are 4E-BP3 blockers. Accordingly, provided herein, in some embodiments, are methods for promoting healthy aging in a human or preventing a disease associated with the elderly in a human comprising administering to the human in need thereof a therapeutically effective amount of a compound of Formula I, or Formula II or Formula III. In some embodiments, a 4E-BP3 blocker suitable for methods described herein is PP242 (2-(4-amino-1-isopropyl-1H-pyrazolo[3,4-d]pyrimidin-3-yl)-1H-indol-5-ol) (Compound 1).

### Chemical Terminology

The term “optionally substituted” or “substituted” means that the referenced group substituted with one or more additional group(s). In certain embodiments, the one or more additional group(s) are individually and independently selected from amide, ester, alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, amido.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “lower alkyl” is a C₁-C₆ alkyl. A “heteroalkyl” group substitutes any one of the carbons of the alkyl group with a heteroatom having the appropriate number of hydrogen atoms attached (e.g., a CH₂ group to an NH group or an O group).

As used herein, the term “aryl” refers to an aromatic ring wherein each of the atoms forming the ring is a carbon atom. Aryl rings described herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

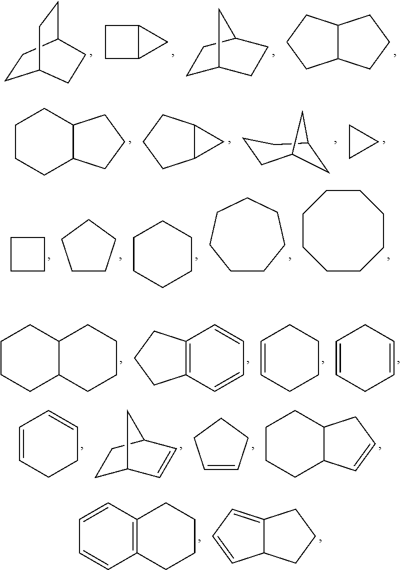

and the like. Monocyclic cycloalkyls include, but are not limited to, cyclopropyl, cyclobutyl, cyclopentyl, cyclohexyl, cycloheptyl, and cyclooctyl. Dicylclic cycloalkyls include, but are not limited to tetrahydronaphthyl, indanyl, tetrahydropentalene or the like. Polycyclic cycloalkyls include admantane, norbornane or the like. The term cycloalkyl includes “unsaturated nonaromatic carbocyclyl” or “nonaromatic unsaturated carbocyclyl” groups both of which refer to a nonaromatic carbocycle, as defined herein, that contains at least one carbon carbon double bond or one carbon carbon triple bond.

The term “heterocyclo” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic or polycyclic. Examples of monocyclic heteroaryl groups include and are not limited to

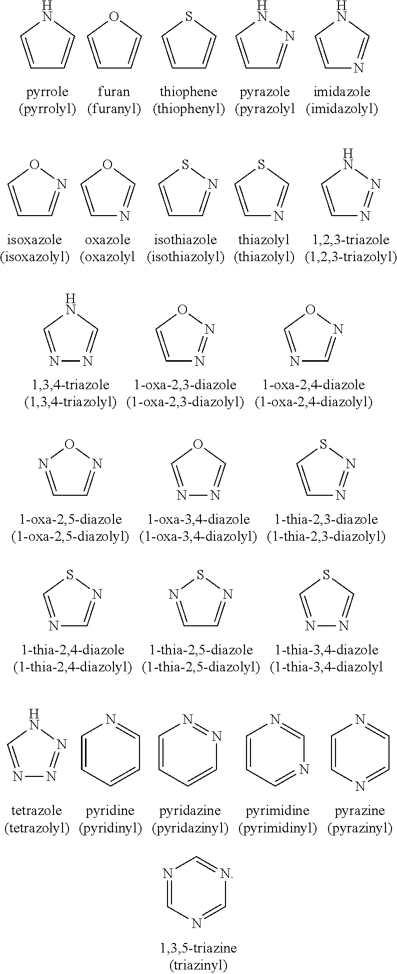

Examples of bicyclic heteroaryl groups include and are not limited to

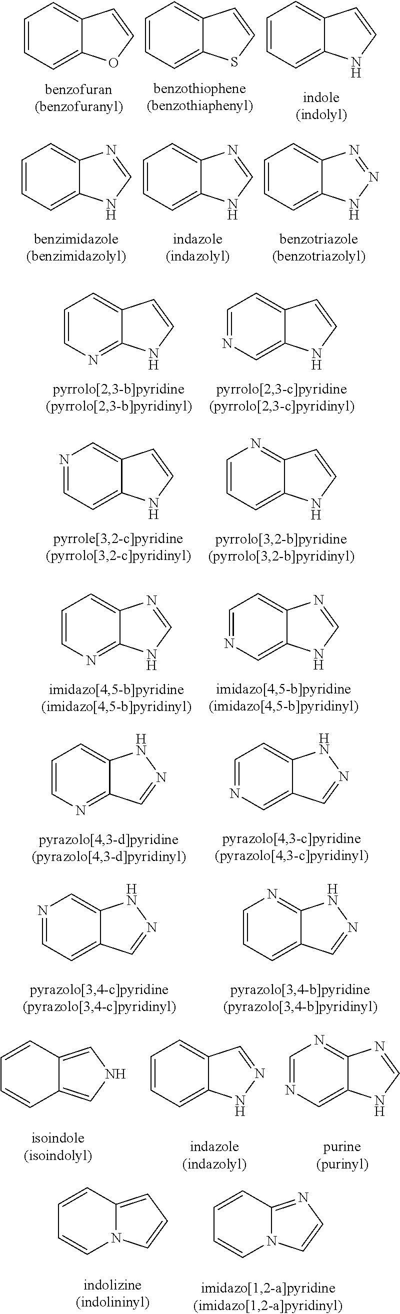

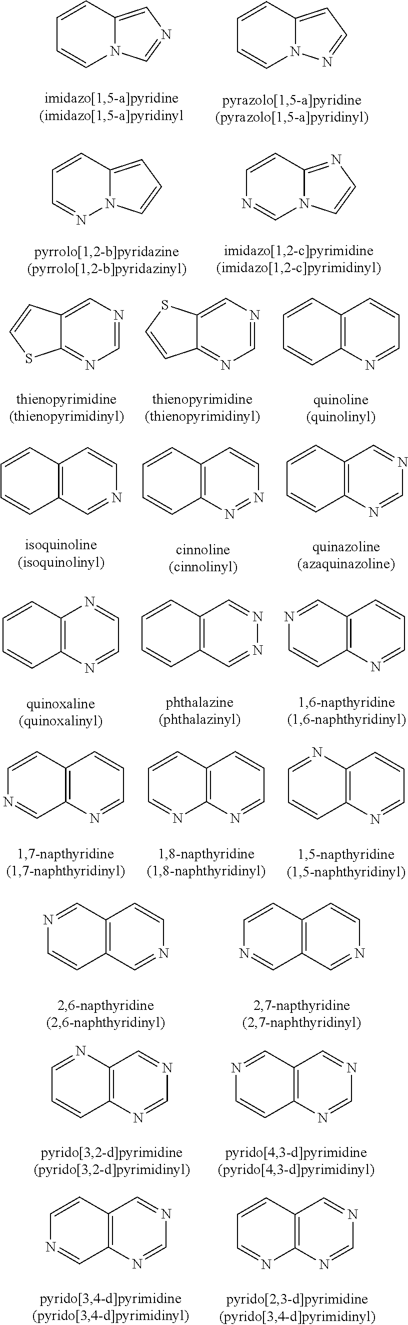

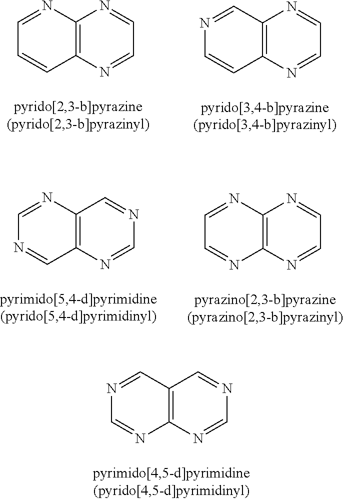

or the like.

A “heteroalicyclic” group or “heterocyclo” group or “heterocycloalkyl” group or “heterocyclyl” group refers to a cycloalkyl group, wherein at least one skeletal ring atom is a heteroatom selected from nitrogen, oxygen and sulfur. In some embodiments, the radicals are fused with an aryl or heteroaryl. Example of saturated heterocyloalkyl groups include

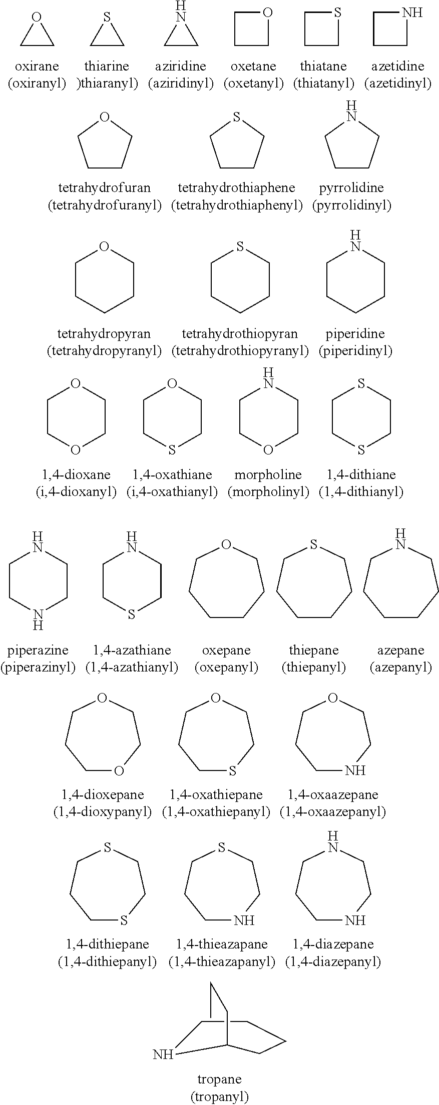

Examples of partially unsaturated heterocyclyl groups include

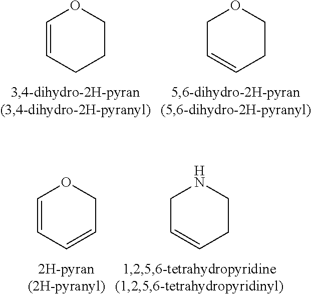

Other illustrative examples of heterocyclo groups, also referred to as non-aromatic heterocycles, include:

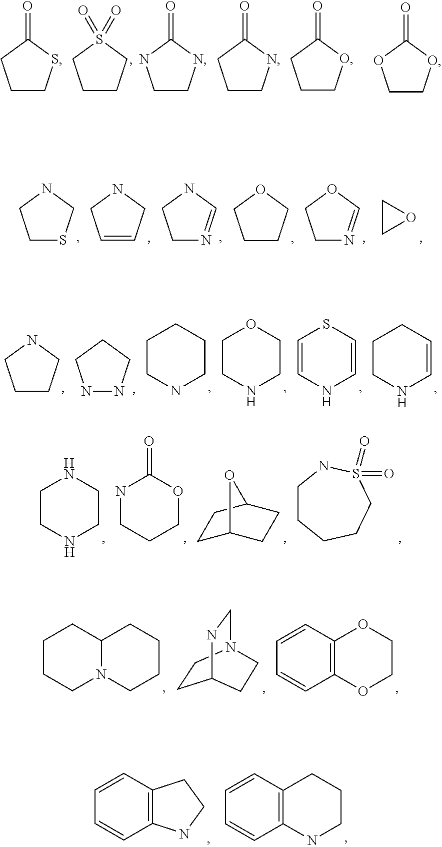

or the like.

The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃.

### Further Details on Compounds

In certain embodiments, compounds described herein have one or more chiral centers. As such, all stereoisomers are envisioned herein. In various embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer are utilized as the therapeutic compound described herein. In certain embodiments, compounds described herein contain one or more chiral centers. These compounds are prepared by any means, including enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-tolunenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosure). General methods for the preparation of compound as described herein are modified by the use of appropriate reagents and conditions, for the introduction of the various moieties found in the formulae as provided herein. As a guide the following synthetic methods are utilized.

Compounds described herein are synthesized starting from compounds that are available from commercial sources or that are prepared using procedures outlined herein.

Formation of Covalent Linkages by Reaction of an Electrophile with a Nucleophile

The compounds described herein are modified using various electrophiles and/or nucleophiles to form new functional groups or substituents. Table A entitled “Examples of Covalent Linkages and Precursors Thereof” lists selected non-limiting examples of covalent linkages and precursor functional groups which yield the covalent linkages. Table A is used as guidance toward the variety of electrophiles and nucleophiles combinations available that provide covalent linakges. Precursor functional groups are shown as electrophilic groups and nucleophilic groups.

TABLE A
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it is necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. In some embodiments it is contemplated that each protective group be removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a Pd⁰-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups are selected from:

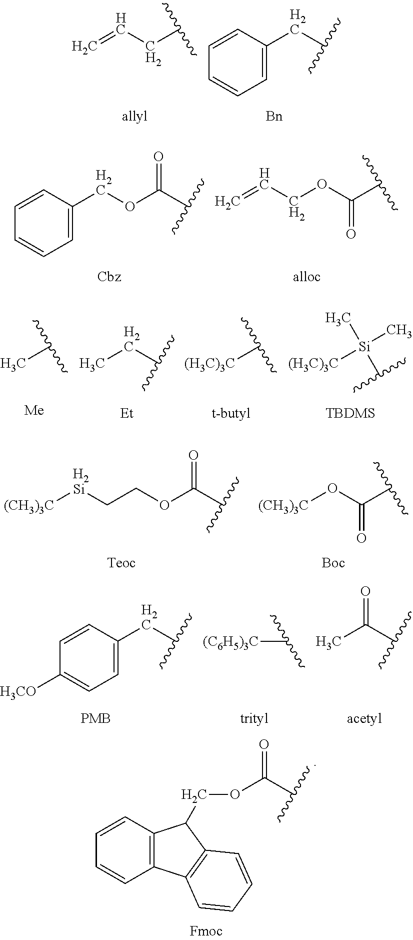

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

Combination Therapy

Described herein are studies that show association of certain genomic regions (e.g., 4EIFBP3 genomic region) in humans with age-related diseases such as cardiovascular disease, metabolic syndrome and/or neurodegenerative disorders. In some embodiments, provided herein are anti-aging methods that prevent or delay onset and/or progression of diseases associated with aging in illderly, wellderly and/or in individuals who are pre-disposed to diseases associated with aging comprising administration of an agent that modulates the (a) activity or expression of at least one gene in the associated genomic region or (b) modulation of the level or activity of at least one expressed product thereof, in combination with a second therapeutic agent. In some embodiments, a second therapeutic agent is an agent that modulates pathways implicated in cell growth, metabolism and/or lifespan.

### Modulators of Insulin-Like Growth Factor (IGF-1) Signaling

In one embodiment, a second therapeutic agent suitable for combination therapy is a modulator of insulin-like growth factor IGF-1 signaling. Examples of modulators of IGF-1 signaling include biguanides such as metformin, phenformin, buformin, proguanil or the like, and compounds described in U.S. Patent Appl. Pub. Nos. 20030130284, 20020045622; modulators of IGF-1 signaling described therein are incorporated herein by reference.

Accordingly, provided herein are anti-aging methods that promote healthy aging and/or prevent or delay onset and/or progression of diseases associated with aging in illderly, wellderly and/or in individuals who are pre-disposed to diseases associated with aging comprising administration of an agent that modulates the (a) activity or expression of at least one gene in an associated genomic region (e.g., EIF4BP3 region) or (b) the level or activity of at least one expressed product thereof, and a modulator of IGF-1 signaling.

In specific embodiments, provided herein are methods for promoting healthy aging and/or preventing or delaying onset and/or progression of diseases associated with aging in illderly, wellderly and/or in individuals who are pre-disposed to diseases associated with aging comprising administration of a 4E-BP3 blocker (e.g., a compound of Formula I, or Formula II or Formula III) and a modulator of IGF-1 signaling.

### Modulators of TOR Signaling Pathways

In some embodiments, a second therapeutic agent suitable for combination therapy is a modulator of TOR signaling. Accordingly, provided herein are anti-aging methods that promote healthy aging and/or prevent or delay onset and/or progression of diseases associated with aging in illderly, wellderly and/or in individuals who are pre-disposed to diseases associated with aging comprising administration of an agent that modulates the (a) activity or expression of at least one gene in an associated genomic region or (b) the level or activity of at least one expressed product thereof, and a modulator of TOR signaling. In some embodiments, a modulator of TOR signaling is a PI-3 kinase inhibitor, an mTOR kinase inhibitor, an AKT inhibitor or any combination thereof. In some embodiments, a modulator of TOR signaling is a PDK 1/2 inhibitor. In some embodiments, a modulator of TOR signaling is a S6K inhibitor.

mTOR (mammalian target of rapamycin) signaling regulates translation and is responsive to nutrients and mitogens such as growth factors. In some instances, the mTOR protein kinase (also known as FRAP) acts as an ATP sensor to regulate cell growth. Growth factor receptors (e.g., IGF-1) first stimulate PI 3 kinase, and through inositol phosphates activate PDK-1 and AKT (protein kinase B). AKT then phosphorylates mTOR. Activation of mTOR, leads to downstream events including phosphorylation of ribosomal protein S6 (S6K1) and 4E-BPs. Phosphorylation of ribosomal protein S6 (S6K1) by S6 Kinase (S6K) stimulates the translation of mRNAs with a 5′ oligopyrimidine tract which encode components of protein synthesis.

Examples of inhibitors of mTOR and/or PI-3 kinase and/or AKT include and are not limited to rapamycin, PX-866, compounds described in U.S. Pat. No. 7,235,555, U.S. Appl. Pub. Nos. 20100068204, 20100061982, 20100056523, 20100029609, 20100015141, 20100003250, 20100003246, 20090318405, 20090311217, 20090304692, 20090298820, 20090291079, 20090263354, 20090227575, 20090209607, 20090192147, 20090162338, 20090169558, 20090098086, 20090048313, mTOR pathway inhibitors described therein are incorporated herein by reference.

Examples of S6K inhibitors include, and are not limited to compounds described in U.S. Patent Appl. Pub. No. 20080234276, the S6K kinase inhibitors described therein are incorporated herein by reference. Examples of inhibitor of PDK 1/2 include BX-424 (Berlex Biosciences); OSU-03012, OSU-03013 (Ohio State University) and compounds described in U.S. Patent Appl. Pub. Nos. 20090209618, 20070286864, the PDK 1/2 inhibitor compounds described therein are incorporated herein by reference.

In specific embodiments, provided herein are methods for promoting healthy aging and/or preventing or delaying onset and/or progression of diseases associated with aging in illderly, wellderly and/or in individuals who are pre-disposed to diseases associated with aging comprising administration of a 4E-BP3 blocker (e.g., a compound of Formula I, or Formula II or Formula III) and a modulator of TOR signaling.

In some of the above embodiments, the 4E-BP3 blocker is administered in combination with an mTOR kinase inhibitor (e.g., rapamycin). In some of the above embodiments, the 4E-BP3 blocker is administered in combination with a PI-3 kinase inhibitor (e.g., PX-866). In some of the above embodiments, the 4E-BP3 blocker is administered in combination with an AKT inhibitor. In some of the above embodiments, the 4E-BP3 blocker is administered in combination with a S6K inhibitor. In some of the above embodiments, the 4E-BP3 blocker is administered in combination with a PDK 1/2 inhibitor. In some of the above embodiments, a 4E-BP3 blocker is a compound of Formula I, Formula II or Formula III (including, e.g., Compound 1).

### Sirtuin Pathway Modulators

In other embodiments, a second therapeutic agent suitable for combination therapy is a modulator of the sirtuin pathway. Accordingly, provided herein are anti-aging methods that promote healthy aging and/or prevent or delay onset and/or progression of diseases associated with aging in illderly, wellderly and/or in individuals who are pre-disposed to diseases associated with aging comprising administration of an agent that modulates the (a) activity or expression of at least one gene in an associated genomic region (e.g., EIF4BP3 region) or (b) the level or activity of at least one expressed product thereof, and a modulator of the sirtuin pathway. In some embodiments, a modulator of the sirtuin pathway is a sirtuin activator.

Examples of Sirtuin activators include trans-stilbene, cis-stilbene, resveratrol, piceatannol, rhapontin, deoxyrhapontin, butein, chalcon; isoliquirtigen; butein; 4,2′,4′-trihydroxychalcone; 3,4,2′,4′,6′-pentahydroxychalcone; flavone, morin, fisetin; luteolin; quercetin; kaempferol; apigenin; gossypetin; myricetin; 6-hydroxyapigenin; 5-hydroxyflavone; 5,7,3′,4′,5′-pentahydroxyflavone; 3,7,3′,4′,5′-pentahydroxyflavone; 3,6,3′,4′-tetrahydroxyflavone; 7,3′,4′,5-tetrahydroxyflavone; 3,6,2′,4′-tetrahydroxyflavone; 7,4′-dihydroxyflavone; 7,8,3′,4′-tetrahydroxyflavone; 3,6,2′,3′-tetrahydroxyflavone; 4′-hydroxyflavone; 5-hydroxyflavone; 5,4′-dihydroxyflavone; 5,7-dihydroxyflavone; daidzein, genistein, naringenin; flavanone; 3,5,7,3′,4′-pentahydroxyflavanone; pelargonidin chloride, cyanidin chloride, delphinidin chloride, (−)-epicatechin (Hydroxy Sites: 3,5,7,3′,4′); (−)-catechin (Hydroxy Sites: 3,5,7,3′,4′); (−)-gallocatechin (Hydroxy Sites: 3,5,7,3′,4′,5′) (+)-catechin (Hydroxy Sites: 3,5,7,3′,4′); (+)-epicatechin (Hydroxy Sites: 3,5,7,3′,4′); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acid.H20); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol.2HCl); β-1′-5-methyl-nicotinamide-2′-deoxyribose; β-D-1′-5 -methyl-nico-tinamide-2′-deoxyribofuranoside; β-1′-4,5-dimethyl-nicotinamide-2′-de-oxyribose; or β-D-1′-4,5-dimethyl-nicotinamide-2′-deoxyribofuranoside; dipyridamole, ZM 336372 (3-(dimethylamino)-N-[3-[(4-hydroxybenzoyl)-amino]-4-met hylphenyl]benzamide), camptothecin, coumestrol, nordihydroguaiaretic acid, esculetin, SRT-1720 (Sirtris), SRT-1460 (Sirtris), SRT-2183 (Sirtris), analogs thereof, or combinations thereof.

In specific embodiments, provided herein are methods for promoting healthy aging and/or preventing or delaying onset and/or progression of diseases associated with aging in illderly, wellderly and/or in individuals who are pre-disposed to diseases associated with aging comprising administration of a 4E-BP3 blocker (e.g., a compound of Formula I, or Formula II or Formula III) and a sirtuin activator.

### Modulators of FOX Transcription Factors

In some embodiments, a second therapeutic agent suitable for combination therapy is a modulator of FOX transcription factors. Accordingly, provided herein are anti-aging methods that promote healthy aging and/or prevent or delay onset and/or progression of diseases associated with aging in illderly, wellderly and/or in individuals who are pre-disposed to diseases associated with aging comprising administration of an agent that modulates the (a) activity or expression of at least one gene in an associated genomic region (e.g., EIF4BP3 region) or (b) the level or activity of at least one expressed product thereof, and a modulator of FOX transcription facators. In some embodiments, a modulator of the FOXO transcription factors is a modulator of the FOXO3 transcription factor.

FOX (Forkhead box) proteins are a family of transcription factors regulate the expression of genes involved in cell growth, stress tolerance and/or lifespan. FOX proteins (transcription factors) include and are not limited to FOXO1, FOXO2, FOXO3, FOXO4, or variants thereof. Examples of modulators of FOXO transcription factors include and are not limited to RNAi.

In specific embodiments, provided herein are methods for promoting healthy aging and/or preventing or delaying onset and/or progression of diseases associated with aging in illderly, wellderly and/or in individuals who are pre-disposed to diseases associated with aging comprising administration of a 4E-BP3 blocker (e.g., a compound of Formula I, or Formula II or Formula III) and a modulator of FOXO3A transcription factors.

Other Agents

In some embodiments, a second therapeutic agent suitable for combination therapy is a modulator of Cdc2 kinase. Accordingly, provided herein are anti-aging methods that promote healthy aging and/or prevent or delay onset and/or progression of diseases associated with aging in illderly, wellderly and/or in individuals who are pre-disposed to diseases associated with aging comprising administration of an agent that modulates the (a) activity or expression of at least one gene in an associated genomic region (e.g., EIF4BP3 region) or (b) the level or activity of at least one expressed product thereof, and a modulator of Cdc2 kinase.

In some embodiments, a second therapeutic agent suitable for combination therapy is a modulator of a MAP kinase. Accordingly, provided herein are anti-aging methods that promote healthy aging and/or prevent or delay onset and/or progression of diseases associated with aging in illderly, wellderly and/or in individuals who are pre-disposed to diseases associated with aging comprising administration of an agent that modulates the (a) activity or expression of at least one gene in an associated genomic region (e.g., EIF4BP3 region) or (b) the level or activity of at least one expressed product thereof, and a modulator of MAP kinases (e.g., JNK inhibitors, ERK inhibitors, p38 inhibitors).

In some embodiments, a second therapeutic agent suitable for combination therapy is a modulator of a product of the EIF4EBP1 or EIF4EBP2 genomic regions. Accordingly, in some embodiments, provided herein are anti-aging methods that promote healthy aging and/or prevent or delay onset and/or progression of diseases associated with aging in illderly, wellderly and/or in individuals who are pre-disposed to diseases associated with aging comprising administration of an agent that modulates the (a) activity or expression of at least one gene in the EIF4EBP3 region or (b) the level or activity of at least one expressed product thereof, and a modulator of a product of at least one gene in the EIF4EBP1 or EIF4EBP2 genomic regions.

In some embodiments, a second therapeutic agent suitable for combination therapy is a modulator of p70S6K ribosomal protein. Accordingly, in some embodiments, provided herein are anti-aging methods that promote healthy aging and/or prevent or delay onset and/or progression of diseases associated with aging in illderly, wellderly and/or in individuals who are pre-disposed to diseases associated with aging comprising administration of an agent that modulates the (a) activity or expression of at least one gene in an associated genomic region (e.g., EIF4BP3 region) or (b) the level or activity of at least one expressed product thereof, and a modulator of p70S6K ribosomal protein (S6K1).

### Examples of Pharmaceutical Compositions and Methods of Administration

Provided herein, in certain embodiments, are compositions comprising a therapeutically effective amount of any compound described herein (e.g., an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III)). Pharmaceutical compositions are formulated using one or more physiologically acceptable carriers including excipients and auxiliaries which facilitate processing of the active compounds into preparations which are used pharmaceutically. Proper formulation is dependent upon the route of administration chosen. A summary of pharmaceutical compositions is found, for example, in Remington: The Science and Practice of Pharmacy, Nineteenth Ed (Eahston, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include one or more agents that (a) modulate the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulate the level or activity of at least one expressed product thereof (e.g., a 4E-BP3 blocker, a compound of Formula I, or Formula II or Formula III) as described herein, and a pharmaceutically acceptable diluent(s), excipient(s), or carrier(s).

In addition, the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III)) is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, with other chemical components, such as carriers, stabilizers, diluents, dispersing agents, suspending agents, thickening agents, and/or excipients. The pharmaceutical composition facilitates administration of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, to an organism. In practicing the methods of treatment or use provided herein, therapeutically effective amounts of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, are administered in a pharmaceutical composition to a mammal having a condition, disease, or disorder to be treated. Preferably, the mammal is a human. A therapeutically effective amount varies depending on the severity and stage of the condition, the age and relative health of an individual, the potency of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, used and other factors. The agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is optionally used singly or in combination with one or more therapeutic agents as components of mixtures.

The pharmaceutical formulations described herein are optionally administered to a individual by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions will include at least one agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these agents that (a) modulate the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulate the level or activity of at least one expressed product thereof (e.g., a 4E-BP3 blocker, a compound of Formula I, or Formula II or Formula III) as described herein, having the same type of activity. In some situations, an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, exists as tautomers. All tautomers are included within the scope of the compounds presented herein. Additionally, the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, exists in unsolvated as well as solvated forms with pharmaceutically acceptable solvents such as water, ethanol, and the like. The solvated forms of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, are also considered to be disclosed herein.

“Carrier materials” include any commonly used excipients in pharmaceutics and should be selected on the basis of compatibility with compounds disclosed herein, such as, an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, a formulation comprising an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is a solid drug dispersion. A solid dispersion is a dispersion of one or more active ingredients in an inert carrier or matrix at solid state prepared by the melting (or fusion), solvent, or melting-solvent methods. (Chiou and Riegelman, Journal of Pharmaceutical Sciences, 60, 1281 (1971)). The dispersion of one or more active agents in a solid diluent is achieved without mechanical mixing. Solid dispersions are also called solid-state dispersions. In some embodiments, any compound described herein (e.g., an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein) is formulated as a spray dried dispersion (SDD). An SDD is a single phase amorphous molecular dispersion of a drug in a polymer matrix. It is a solid solution prepared by dissolving the drug and a polymer in a solvent (e.g., acetone, methanol or the like) and spray drying the solution. The solvent rapidly evaporates from droplets which rapidly solidifies the polymer and drug mixture trapping the drug in amorphous form as an amorphous molecular dispersion. In some embodiments, such amorphous dispersions are filled in capsules and/or constituted into oral powders for reconstitution. Solubility of an SDD comprising a drug is higher than the solubility of a crystalline form of a drug or a non-SDD amorphous form of a drug. In some embodiments of the methods described herein, agents that (a) modulate the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulate the level or activity of at least one expressed product thereof (e.g., a 4E-BP3 blocker, a compound of Formula I, or Formula II or Formula III) as described herein, are administered as SDDs constituted into appropriate dosage forms described herein.

Pharmaceutical preparations for oral use are optionally obtained by mixing one or more solid excipient with an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active compound doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

The pharmaceutical solid oral dosage forms including formulations described herein, which include an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, are optionally further formulated to provide a controlled release of the agent. Controlled release refers to the release of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a individual over an extended period of time according to a predetermined profile. Such release rates provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein, which include an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, are delivered using a pulsatile dosage form. A pulsatile dosage form is capable of providing one or more immediate release pulses at predetermined time points after a controlled lag time or at specific sites. Pulsatile dosage forms including the formulations described herein, which include an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are elf-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is optionally in a form such as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, and a suitable powder base such as lactose or starch.

Buccal formulations that include an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) a formulation of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is optionally accomplished by means of iontophoretic patches and the like. Additionally, transdermal patches provide controlled delivery of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein. The rate of absorption is optionally slowed by using rate-controlling membranes or by trapping the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, optionally with carriers, optionally a rate controlling barrier to deliver the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, to the skin of the host at a controlled and predetermined rate over a prolonged period of time, and means to secure the device to the skin.

Formulations that include an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, in water soluble form. Additionally, suspensions of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, are optionally prepared as appropriate oily injection suspensions.

In some embodiments, the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

The agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

The agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof, is optionally used in the preparation of medicaments for promoting healthy aging and/or for the prophylactic and/or therapeutic treatment of an age-related disease that would benefit, at least in part, from amelioration of symptoms.

In addition, any method of treatment described herein, involves administration of pharmaceutical compositions containing at least one agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III), described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

In the case wherein the patient's condition does not improve, upon the doctor's discretion the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III), is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III), is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical compositions described herein are in unit dosage forms suitable for single administration of precise dosages. In unit dosage form, the formulation is divided into unit doses containing appropriate quantities of one or more agents that that (a) modulate the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulate the level or activity of at least one expressed product thereof (e.g., a 4E-BP3 blocker, a compound of Formula I, or Formula II or Formula III),. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The daily dosages appropriate for the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III), are from about 0.001 to 2.5 mg/kg per body weight. An indicated daily dosage in the larger mammal, including, but not limited to, humans, is in the range from about 0.1 mg to about 1000 mg, conveniently administered in divided doses, including, but not limited to, up to four times a day or in extended release form. Suitable unit dosage forms for oral administration include from about 1 to 500 mg active ingredient, from about 1 to 250 mg of active ingredient, or from about 1 to about 100 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of the agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III), the disease or condition to be treated, the mode of administration, the requirements of an individual, the severity of the disease or condition being treated, and the judgment of the practitioner.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. Agents that exhibit high therapeutic indices are preferred. The data obtained from cell culture assays and animal studies is optionally used in formulating a range of dosage for use in human. The dosage of such agents that (a) modulate the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulate the level or activity of at least one expressed product thereof (e.g., a 4E-BP3 blocker, a compound of Formula I, or Formula II or Formula III), lies preferably within a range of circulating concentrations that include the ED50 with minimal toxicity. The dosage optionally varies within this range depending upon the dosage form employed and the route of administration utilized.

Assays for Identification of Agents that Modulate 4E-BPs

In some embodiments, agents that modulate 4E-BPs are identified by use of in vitro assays. By way of example, an in vitro assay for a chemical-genetic screen for identification of 4E-BP modulators is described by Livingstone et al. in Chem Biol. 2009 Dec 24;16(12):1240-9. The chemical-genetic assay described by Livingstone et al., is based on determination of the nuclear accumulation of eIF4E, which occurs in a 4E-BP-dependent manner specifically upon inhibition of mTOR signaling, and the disclosure of the assay is incorporated herein by reference. In some embodiments, the chemical-genetic assays are adapted for high-throughput screening (HTS) using any suitable method.

### EXAMPLES

### Materials and Methods

### Sample

All samples from study participant samples were collected using a written informed consent, and sample collections were approved by local study institutional review boards.

Individuals who were at least 80 years of age and had no chronic diseases and were not taking any significant chronic medications were enrolled in the study. For a suitable control group, DNA from a group of individuals who had died of chronic diseases, matched for birth year, gender, and ancestry was used. The finding was replicated via a second cohort of such individuals

### Example 1

### Genotyping

A total of 397 unique Wellderly, 386 Illderly, and 1128 young population control DNA samples were genotyped using the Illumina whole-genome genotyping BeadChips as outlined in Table 1. Genotypes were called using a clustering algorithm in Illumina's Genome Studio software. Samples with average call rates <98% were removed and SNPs were re-clustered. SNPs with average call rates <95% were discarded, and SNPs with cluster separation score <0.25, Hardy-Weinberg P<10-5, or heterozygote genotype frequencies >0.65 were manually inspected and cluster positions were edited if needed. Prior to analysis, SNPs with a minor allele frequency <0.05 and Hardy Weinberg Equilibrium P<0.0001 were excluded. The final number of SNPs and quality metrics are shown in Table 8.

Forty-six top SNPs from the initial analysis of the GWAS phase of the study were genotyped in the Wellderly and Illderly samples using Sequenom MassArray genotyping platform to confirm genotyping, and also in a second set of 284 wellderly samples for replication. Polymerase chain reaction (PCR) assays and extension primers for these SNPs were designed using the MassARRAY Assay Design software, version 3.1 (Sequenom). SNPs were genotyped using the iPLEX Gold assay, based on multiplex PCR followed by a single base primer extension reaction. The mass of the primer extension products, correlating to genotype, were determined using Matrix Assisted Laser Desorption/Ionization Time-of-Flight (MALDI-TOF) Mass Spectrometry. Final genotypes were called using the MassArray Type, version 4.0.

### Example 2

### Genome-Wide Association

A Genome-wide association study was carried out using methods implemented in PLINK (http://pngu.mgh.harvard.edu/purcell/plink/; 24). To start, SNPs were analyzed using a chi-square test to compare case and control allele frequencies. Both the wellderly-illdery and wellderly-young controls showed relatively high evidence for population stratification (λ=1.08 for both analyses, respectively). Therefore population outliers and estimated population stratification for each population using multidimensional scaling (MDS) as implemented in PLINK, including samples from the HapMap 3 populations (hapmap3_r2_b36 release) were determined. A subset of 52,316 autosomal markers were used that were in linkage equilibrium (window size 50, shift window at 5 SNP increments, variance inflation factor 2), and had 100% call sample call rates. Twenty wellderly, 4 illderly, and 168 young population control individuals were removed due to being outliers because they clustered outside of HapMap individuals with European ancestry (CEU and TSI populations). All subsequent analyses were conducted using logistic regression and the first 10 MDS components and gender as covariates. The removal of outliers and adjusting for the first 10 MDS components removed evidence for population stratification (λ=1.017 and 1.025 for wellderly-illderly and wellderly-young controls, respectively). For the genome-wide association study of BMI in the wellderly, a linear regression model was used with the first 10 MDS component and gender as covariates.

### Study Design

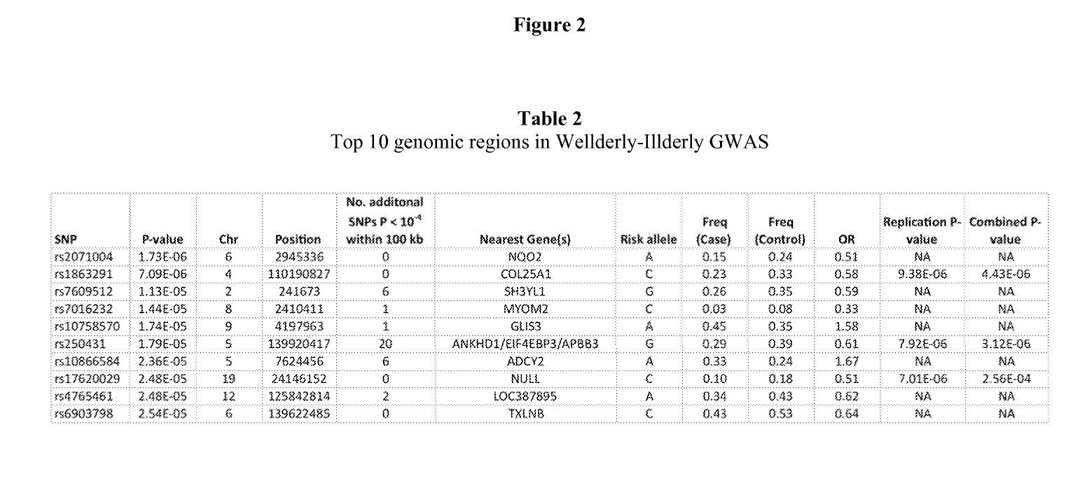

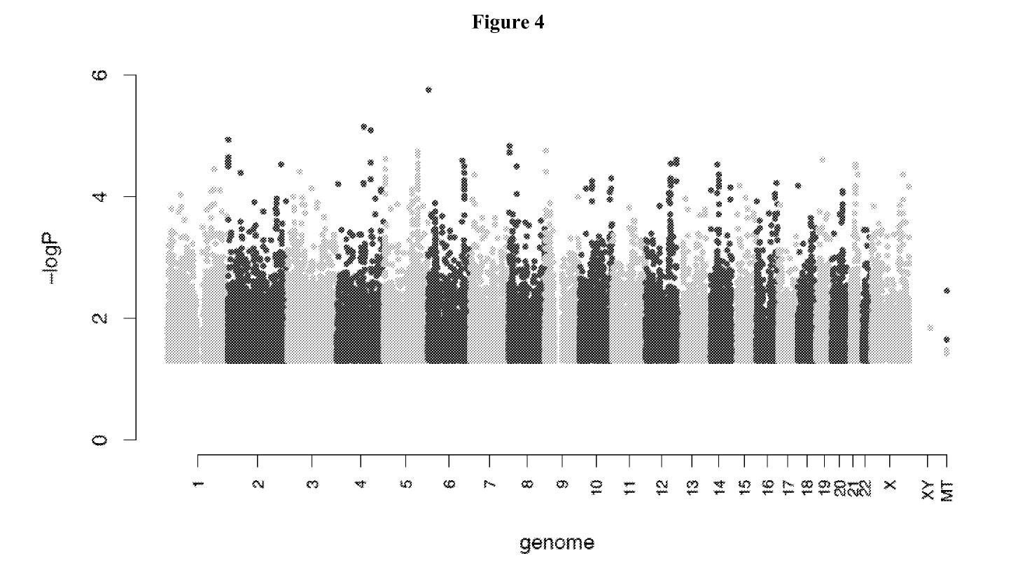

The overall study design is shown in Table 1. The initial genome-wide association study was conducted in a set of 397 wellderly cases and 386 illderly controls, both genotyped using the Illumina Human1M Genotyping BeadChip. We followed up a subset of associated SNPs from the initial analysis in a second set of 284 wellderly samples and compared to the same set of 386 illderly controls. The results of the initial genome-wide association study of the wellderly-illderly sample are shown in FIG. 4. Table 2 shows the top 10 genomic regions identified from the wellderly-illderly genome-wide association study. We were drawn to the genomic region containing EIF4EBP3 as it was in one of the most highly associated genomic regions, and has 21 SNPs in the region that are associated (P<10−4), lending more evidence that the result is a true association. FIG. 2 shows the EIF4EBP3 gene region in more detail, with the associated region spanning from genes SLC4A9 to APPB3, encompassing the additional genes ANKHD1, ANKHD1-EIF4EBP3, and SRA1. The most highly associated SNP in the EIF4EBP3 gene region is rs250431, a coding (synonymous) SNP in APPB3. The genomic region has marked strong linkage disequilibrium with an LD block spanning approximately 199.8 kb from rs34507975 to rs250431.

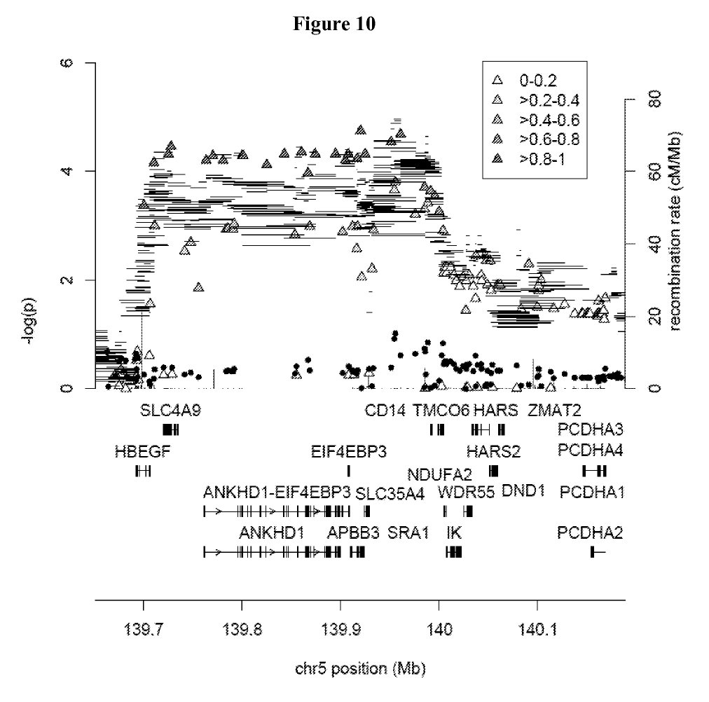

For the associated region in EIF4EBP3 gene region, a sliding window haplotype analysis was conducted using window lengths from 2 to 10 SNPs. The P-values for the OMNIBUS association (overall association for all haplotypes within a window) are shown in FIG. 10 and are similar to the single locus P-values indicating that the associated allele from the most associated rs250431 is on a single haplotype. In addition, we tested the association of the genome accounting for the most associated SNP in the EIF4EBP3 gene region, rs250431 (FIG. 10). No additional signals in the EIF4EBP3 gene region are present, indicating that the association is not being driven by additional independent SNPs in the EIF4BP3 gene region.

We genotyped rs250431 in a second set of 284 wellderly samples and compared to the same set of illderly controls, and the result was significant (P=7.92×10−6). We then combined all 681 wellderly samples and re-analyzed comparing the full set of wellderly case samples to the 386 illderly control samples (P=3.12×10−6). The results for additional SNPs in other genomic regions are shown in Table 2.

Since the allele frequency of the most highly associated SNP in the EIF4EBP3 gene region was higher in the illderly versus the wellderly groups (0.39 and 0.29, respectively) we investigated the allele frequency of this SNP in HapMap CEU (European ancestry). The allele frequency is 0.22, indicating that the association is largely being driven by over-representation of the variant in the illderly control group rather than the wellderly case group. In addition, we looked at the allele frequency of this SNP in the healthy young European ancestry sample. Although this SNP was not directly genotyped in the healthy young sample, two SNPs that are perfect proxies to rs250431 in the HapMap CEU data, rs250426 and rs250429, were genotyped and have an allele frequency of 0.31.

This locus has two rare events. A gene fusion exists between ANKDH1 and EIF4EBP3 and the use of an alternative reading frame is rare. All known SNPs+/−2 kb of the genomic region containing the ANKDH1-EIF4EBP3 read-through gene were functionally annotated with PupaSuite and FASTSNP. Four likely functional nonsynonymous SNPs rs3178003 (K1461I), rs3172957(K1732Q) rs1051309 (G1585S), and rs3752704 (N17595) were initially identified as damaging by FASTSNP and by codon selection measures implemented in Pupasuite. Of these SNPs, rs3178003 acts as a premature stop codon in a particular transcript of the ANKDH1 gene, ENST00000246149. However, this transcript is not known to read-through to EIF4EBP3 and may not impact EIF4EBP3 function. The remaining SNPs (rs1051309, rs3172957, and rs3752704) were annotated with a number of different coding and splicing functional predictors by F-SNP [Table 4]. The SNP rs3172957 was almost universally predicted to be damaging by all the functional assessment algorithms.

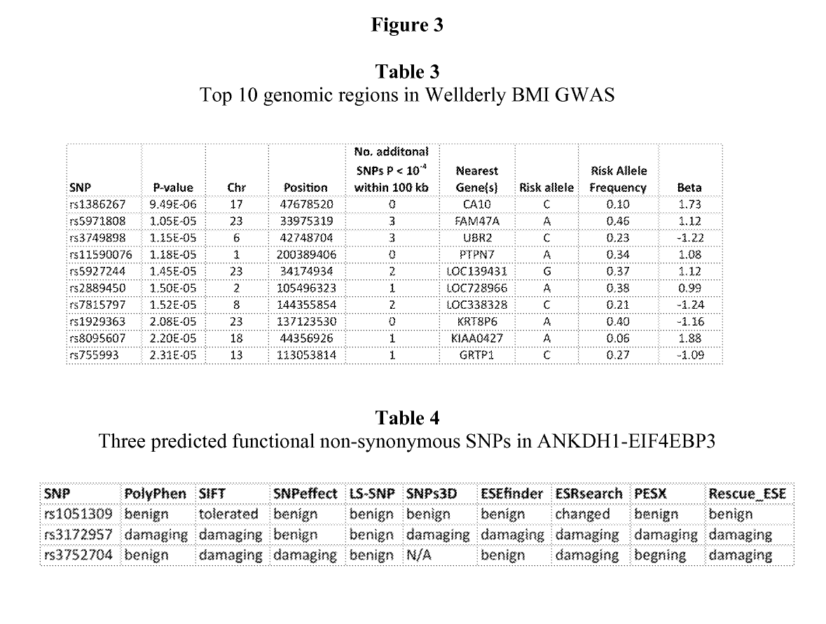

Since there is a strong relationship between dietary restriction and aging and longevity, and there is evidence in our wellderly-illderly analysis that implicates EIF4EBP3 in the mTOR signaling pathway, we looked at the association of body mass index (BMI) in the wellderly cohort. The results of this GWAS are shown in FIG. 3 and Table 3. The third most associated region, which is on chromosome 6 has 4 SNPs with P<10−4 (most highly associated SNP rs3749898, P=1.15−10−5). This SNP is in the intron of the gene UBR2, an E3 ubiquitin ligase that may be a cellular target of leucine. This gene is of interest as leucine enhances mTOR-mediated phosphorylation of S6K1 and 4E-BP.

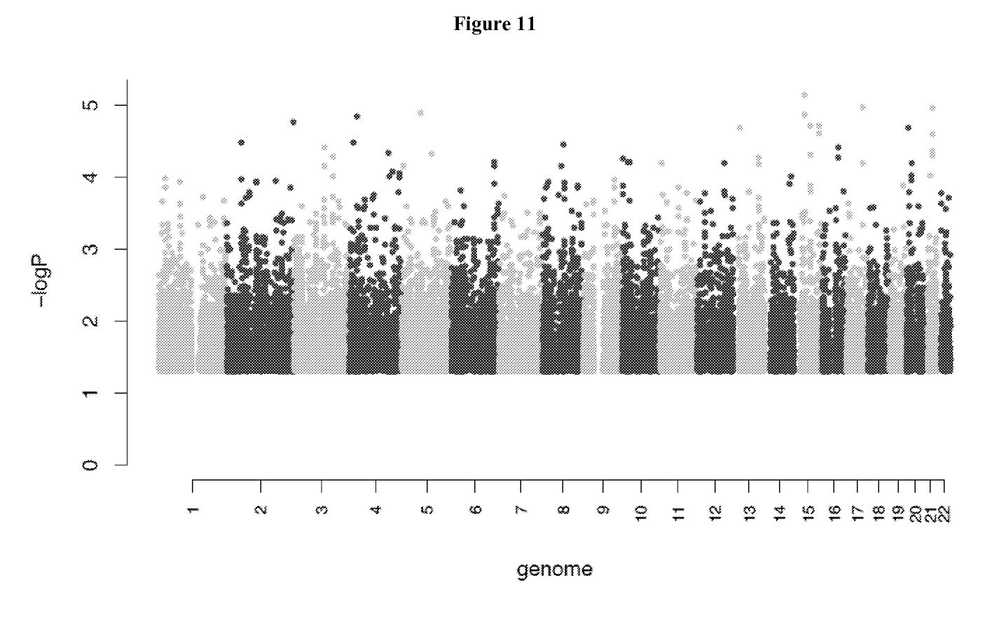

Finally, we compared the wellderly group to a collection of population-based young healthy controls. The genome-wide plots and top 10 genomic regions are shown in FIG. 11 and Table 5, respectively.

### Example 3

### In Vivo Animal Model of Obesity

A non-genetic mouse obesity model is used. Obesity is induced in a cohort of C57BL/6 (wild-type) mice, by a high-fat cafeteria diet. The mice are administered 30 mg/kg of compound 1 once daily as an oral solution in saline. The mice are weighed daily over a period of two weeks. Cafeteria dieting is continued throughout the experiment. Obese mice administered a saline solution alone (placebo) serve as negative controls. The weight gain in the control group is compared with the weight gain or loss of the test group.

### Example 4

### Clinical Trial for Diabetes and Obesity

This is a Phase II study to assess the effect on weight loss and weight maintenance over a period of one year when prescribed with a daily dose of Compound I in obese patients with Type 2 Diabetes. Patients are administered a daily oral dose of Compound 1 for the duration of the study.

Primary Outcome Measures: Change in body weight

Secondary Outcome Measures: Metabolic parameters

Safety, dose-tolerance, glucose levels in blood/plasma, weight loss and HBA1c will be monitored for the duration of the study.

Eligibility: 55-80 years; BMI must be greater than 27 and less than 40; Treated Type 2 Diabetes on single therapy treatment for at least 1 year; Stable weight (variation of less than 5 kg within 3 months prior to screening visit)

### Example 5

### Clinical Trial for Alzheimer's Disease

The following human clinical trial is performed to determine the safety and efficacy of Compound 1 for the treatment of Alzheimer's disease. The study aims to provide preliminary estimates of effect of administration of Compound 1 in delaying progression of disease over a study period of one year.

Sixty patients between the ages of 55 and 80 are recruited via referrals from hospitals, after the patients have been diagnosed with mid stage Alzheimer's disease using the Mini-Mental State Exam scores and a clinical interview.

A screening visit is arranged and a full explanation of the study prior to screening is provided if the patient appeared suitable for and interested in taking part. For inclusion, all patients are required to meet the following criteria: (i) diagnosis of Alzheimer's disease (ii) a study partner who can attend all study visits (iii) negative urine screening for illicit drugs (iv) cooperative, able to ingest oral medication and willing to undertake repeated cognitive testing, (v) able to provide written informed consent. Exclusion criteria include (i) significant neurological disease other than Alzheimer's disease (ii) significant depression or other psychiatric disorder (iii) unstable medical conditions. The study procedures are approved by an institutional ethics review board. All patients in the study must provide written informed consent.

After screening has identified suitable patients that have provided informed consent, patients are placed on a single-blind placebo for 1 week. After 1 week on placebo (baseline), all patients complete a comprehensive cognitive test battery and undergo clinical assessments, and then are randomized into a double-blind protocol so that, half of the sample received Compound 1 capsules and the remaining half received placebo for the next 52 weeks. Cognitive and clinical assessments are carried out again at 12 weeks, 26 weeks and 52 weeks.

Patients assigned to the Compound 1 group will receive a dose twice a day for 12 weeks at increasing doses. Cognitive assessments for all patients are on the maximum dose. The placebo group will receive identical appearing capsules containing ascorbic acid (100 mg).

The cognitive battery includes measures of executive functioning, verbal skills, verbal and spatial working memory, attention and psychomotor speed. The battery is administered to all patients on all three occasions in the same fixed order (e.g., Mini-Mental State Examination (MMSE), MATRICS cognitive battery, BACS score, and Alzheimer's disease Assessment Scale—Cognitive Subscale (ADAS-Cog)). Patients are allowed to take breaks as needed in order to obtain maximal performance at all times. Tests are administered and scored by trained psychologists who are blind to patients' group affiliations and are not involved in patients' treatment plan in any way. Alzheimer's disease Cooperative Study—Activities of Daily Living (ADCS-ADL) is also recorded.

Patients are told that the aim of the study is to investigate the cognitive effects of Compound 1. They are requested to abstain from alcohol for at least 24 h prior to their scheduled cognitive testing.

The patients in the Compound 1 and placebo groups are compared on demographic, clinical, and cognitive variables obtained at baseline using independent sample I-tests.

The effects of Compound 1 on Neuropsychological Test Battery and Neuropsychiatric Inventory (NPI) are analyzed (separately) by 2 (Treatment: Compound 1, placebo)×3 (Time: baseline, 12 weeks, 26 weeks, 52 weeks) analysis of variance (ANOVA).

All cognitive variables are first examined for their distribution properties, i.e., to ensure normality. The cognitive effects of Compound 1 over time are then evaluated by Treatment×Time ANOVA, performed separately for each variable, with Time as a within-individuals factor and Treatment as a between-individuals factor, followed by post-hoc mean comparisons wherever appropriate. All cognitive effects are then re-evaluated using ANOVA performed separately on change scores computed for each variable (12 weeks data minus baseline data, 26 weeks, 52 weeks data minus baseline data). Alpha level for testing significance of effects is p=0.05.

Primary outcome measure is an improvement in (ADAS-Cog) scores. Secondary outcome measures are improvement in (MMSE) socres and (ADCS-ADL).

### Example 6

### Pharmaceutical Compositions

### Example 6a

### Parenteral Composition

To prepare a parenteral pharmaceutical composition suitable for administration by injection, 100 mg of a water-soluble salt of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is dissolved in DMSO and then mixed with 10 mL of 0.9% sterile saline. The mixture is incorporated into a dosage unit form suitable for administration by injection.

### Example 6b

### Oral Composition

To prepare a pharmaceutical composition for oral delivery, 100 mg of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is mixed with 750 mg of starch. The mixture is incorporated into an oral dosage unit for, such as a hard gelatin capsule, which is suitable for oral administration.

### Example 6c

### Sublingual (Hard Lozenge) Composition

To prepare a pharmaceutical composition for buccal delivery, such as a hard lozenge, mix 100 mg of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is mixed with 420 mg of powdered sugar, with 1.6 mL of light corn syrup, 2.4 mL distilled water, and 0.42 mL mint extract. The mixture is gently blended and poured into a mold to form a lozenge suitable for buccal administration.

### Example 6d

### Fast-Disintegrating Sublingual Tablet

A fast-disintegrating sublingual tablet is prepared by mixing 48.5% by weight of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, 44.5% by weight of microcrystalline cellulose (KG-802), 5% by weight of low-substituted hydroxypropyl cellulose (50 μm), and 2% by weight of magnesium stearate. Tablets are prepared by direct compression (AAPS PharmSciTech. 2006; 7(2):E41). The total weight of the compressed tablets is maintained at 150 mg. The formulation is prepared by mixing the amount of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, with the total quantity of microcrystalline cellulose (MCC) and two-thirds of the quantity of low-substituted hydroxypropyl cellulose (L-HPC) by using a three dimensional manual mixer (lnversina®, Bioengineering AG, Switzerland) for 4.5 minutes. All of the magnesium stearate (MS) and the remaining one-third of the quantity of L-HPC are added 30 seconds before the end of mixing.

### Example 6e

### Inhalation Composition

To prepare a pharmaceutical composition for inhalation delivery, 20 mg of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is mixed with 50 mg of anhydrous citric acid and 100 mL of 0.9% sodium chloride solution. The mixture is incorporated into an inhalation delivery unit, such as a nebulizer, which is suitable for inhalation administration.

### Example 6f

### Rectal Gel Composition

To prepare a pharmaceutical composition for rectal delivery, 100 mg of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 6g

### Topical Gel Composition

To prepare a pharmaceutical topical gel composition, 100 mg of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topic1 administration.

### Example 6h

### Ophthalmic Solution Composition

To prepare a pharmaceutical opthalmic solution composition, 100 mg of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is mixed with 0.9 g of NaCl in 100 mL of purified water and filterd using a 0.2 micron filter. The resulting isotonic solution is then incorporated into ophthalmic delivery units, such as eye drop containers, which are suitable for ophthalmic administration.

### Example 6i

### Nasal Spray Solution

To prepare a pharmaceutical nasal spray solution, 10 g of an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof (e.g., a 4E-BP3 blocker, a compound of Formula I, or Formula II or Formula III) as described herein, is mixed with 30 mL of a 0.05M phosphate buffer solution (pH 4.4). The solution is placed in a nasal administrator designed to deliver 100 μl of spray for each application.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of promoting healthy aging in a human in need thereof or preventing a disease associated with the elderly in a human in need thereof comprising administering to the human an agent that (a) modulates the activity or expression of at least one gene in an illderly- or wellderly-associated genomic region spanning from position 139710628 to 139986614 base pairs on human chromosome 5 based on hg19, GRCh37 Genome Reference Consortium Human Reference 37, or (b) modulates the level or activity of at least one expressed product thereof.

2. The method of claim 1, wherein the disease associated with the elderly is selected from a cardiovascular disease, a metabolic syndrome, a bone-loss disorder, or a neurodegenerative disease.

3. The method of claim 1, wherein the disease associated with the elderly is selected from the group consisting of pre-diabetes, diabetes, obesity, osteoporosis, coronary artery disease, cerebrovascular disease, heart attack, stroke, peripheral arterial disease, aortic valve disease, stroke, mild cognitive impairment, pre-dementia, dementia, macular degeneration, and cataracts.

4. The method of claim 1, wherein the sequence responsible for the expression of a gene contains at least one illderly- or wellderly-associated single nucleotide polymorphism (SNP).

5. The method of claim 4, wherein the illderly- or wellderly-associated SNP is found in the region spanning from genes SLC4A9 to SLC35A4, the region encompassing the gene APPB3, the region encompassing the gene ANKHD1-EIF4EBP3, the region encompassing the gene EIF4BP3, the region encompassing the gene ANKHD1, the region encompassing the gene SLC4A9, the region encompassing the gene SLC35A4, or the region encompassing the gene SRA1.

6. The method of claim 4, wherein the illderly- or wellderly-associated SNP is selected from the group consisting of rs1862176, rs6879217, rs34507975, rs6860077, rs11168051, rs17286676, rs13171154, rs35309382, rs6872579, rs7356554, rs975243, rs1432959, rs12717986, rs10042299, rs12717988, rs7715360, rs7701365, rs3822328, rs10476907, rs1019933, rs10041762, rs9687753, rs12109242, rs6876865, rs6871703, rs7712617, r56579768, rs3733681, rs9324644, rs250432, rs250424, rs250425, rs801460, rs801459, rs10463297, rs250426, rs2249213, rs250431, rs250430, rs250429, rs2253378, rs4912715, rs801458, rs4279384, rs1864255, rs2569163, rs1835148, rs7444290, rs778583, rs778584, rs7721577, and rs778587.

7. The method of claim 4, wherein the illderly- or wellderly-associated SNP is selected from the group consisting of rs250431, rs2071004, rs1863291, rs7609512, rs7016232, rs10758570, rs10866584, rs17620029, rs4765461, rs6903798, rs1386267, rs5971801, rs3749898, rs11590076, rs5927244, rs2889450, rs7815797, rs1929363, rs8095607, rs755993, rs1051309, rs3172957, and rs3752704.

8. The method of claim 1, wherein the at least one gene is selected from the group consisting of SLC4A9, ANKHD1-EIF4EBP3, APPB3, ANKHD1, EIF4EBP3, SRA1 and SLC35A4.

9. The method of claim 1, wherein the at least one gene is EIF4EBP3.

10. The method of claim 1, wherein the at least one expressed product is a protein.

11. The method of claim 10, wherein the protein is a 4E-BP.

12. The method of claim 11, wherein the 4E-BP is 4E-BP3.

13. The method of claim 1, wherein the agent is a 4E-BP3 blocker.

14. The method of claim 1, wherein the agent
(a) reduces or inhibits phosphorylation of 4E-BP3; and/or
(b) increases or promotes binding of 4E-BP3 to eIF4E.

15. The method of claim 14, wherein the agent that reduces or inhibits phosphorylation of 4E-BP3 is an inhibitor or partial inhibitor of a kinase selected from the group consisting of Akt/PI-3 kinase, mTOR kinase, a MAP kinase, a S6 kinase, and Cdc2 kinase.

16. The method of claim 1, wherein the agent is administered in combination with a modulator of IGF, FOXO3, AKT, TOR, or Sirtuin.

17. The method of claim 1, wherein the human in need thereof has a SNP selected from rs1862176, rs6879217, rs34507975, rs6860077, rs11168051, rs17286676, rs13171154, rs35309382, rs6872579, rs7356554, rs975243, rs1432959, rs12717986, rs10042299, rs12717988, rs7715360, rs7701365, rs3822328, rs10476907, rs1019933, rs10041762, rs9687753, rs12109242, rs6876865, rs6871703, rs7712617, rs6579768, rs3733681, rs9324644, rs250432, rs250424, rs250425, rs801460, rs801459, rs10463297, rs250426, rs2249213, rs250431, rs250430, rs250429, rs2253378, rs4912715, rs801458, rs4279384, rs1864255, rs2569163, rs1835148, rs7444290, rs778583, rs778584, rs7721577, and rs778587.

18. The method of claim 1, wherein the agent is a compound having structure:

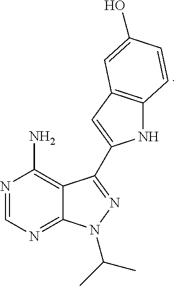

19. A method for predicting a risk for unhealthy aging in a human comprising
(a) detecting, in a sample obtained from said human, at least one illderly-associated single nucleotide polymorphism (SNP), in the genomic region spanning from position 139710628 to 139986614 base pairs on human chromosome 5; and
(b) correlating the detection of the at least one illderly-associated SNP in said human to risk of unhealthy aging;
wherein said detecting is carried out on a Genome Analyzer.

20. The method of claim 19, wherein the illderly-associated SNP is in the genomic region EIF4EBP3.

21. The method of claim 19, wherein the illderly-associated SNP is selected on the basis of its p value of association with illderly versus the wellderly, allele frequency and/or odds ratio in an illderly population.
